(19) United States
(12) Patent Application Publication
(10) Pub. No.: US 2026/0078395 A1
(43) Pub. Date: Mar. 19, 2026

(54) COMPOSITIONS AND METHODS FOR PROTEIN PRODUCTION

(71) Applicant: VELOZBIO COMPANY, Wilmington, DE (US)

(72) Inventors: Laura Margarita LÕPEZ CASTILLO, Wilmington, DE (US); Luis Flavio SILLER RODRIGUEZ, Wilmington, DE (US); Nicole Alejandra MORVELI FLORES, Wilmington, DE (US); Jessica Paola MÉNDEZ CHAVERO, Wilmington, DE (US); Enrique Javier GONZALEZ GONZALEZ, Wilmington, DE (US)

(21) Appl. No.: 19/354,574

(22) Filed: Oct. 9, 2025

Related U.S. Application Data
(63) Continuation of application No. 19/213,842, filed on May 20, 2025.
Application No. 19/213,842 is a continuation of application No. PCT/US23/81674, filed on Nov. 29, 2023.
(60) Provisional application No. 63/385,424, filed on Nov. 30, 2022.
(60) Provisional application No. 63/385,429, filed on Nov. 30, 2022.

(51) Int. Cl. C12N 15/82; C07K 14/47; C07K 14/495

(52) U.S. Cl. CPC C12N 15/8257; C07K 14/4732; C07K 14/495; C12N 15/8205; C12N 15/8207; C12N 15/8251; C07K 2319/21

(57) ABSTRACT

Provided herein are methods for generating a protein product, comprising: providing a plant seed; contacting said plant seed with a protein expression vector that encodes for a protein; transiently expressing said protein in said plant seed using said protein expression vector, and isolating said protein from said plant seed to obtain a protein product.

### CROSS-REFERENCE

This application is a continuation of U.S. Non-Provisional application Ser. No. 19/213,842, filed May 20, 2025, which is a continuation application of PCT/US23/81674, filed Nov. 29, 2023, which claims priority to U.S. Provisional Patent Application No. 63/385,429, filed Nov. 30, 2022, and U.S. Provisional Patent Application No. 63/385,424, filed Nov. 30, 2022, which applications are herein incorporated by reference in their entireties for all purposes.

### SEQUENCE LISTING

The instant application contains a Sequence Listing which has been submitted electronically in XML format and is hereby incorporated by reference in its entirety. Said XML copy, created on Oct. 10, 2025, is named 67278-701_302_SL.xml and is 30,559 bytes in size.

### BACKGROUND

Proteins can be expressed in a variety of systems, including in plants and plant seeds. Different protein expression systems can be used for different proteins. Additionally, proteins can be expressed stably or transiently.

### SUMMARY

Recognized herein is a need for improved compositions and methods for producing proteins.

In an aspect, the present disclosure provides a method for generating a protein product, comprising: (a) providing a legume seed; (b) contacting the legume seed with a protein expression vector that encodes for a protein; (c) transiently expressing the protein in the legume seed using the protein expression vector; and (d) isolating the protein from the legume seed to obtain a protein product.

In some embodiments, the legume seed is selected from the group consisting of a dry bean seed, a dry broad bean seed, a dry pea seed, a chickpea seed, a dry cowpea seed, a pigeon pea seed, a lentil seed, a bambara groundnut seed, a vetch seed, a lupin seed, a pulse NES seed, and a combination thereof. In some embodiments, the legume seed is a dry bean seed. In some embodiments, the dry bean seed is selected from the group consisting of a kidney bean seed, a navy bean seed, a pinto bean seed, a black turple bean seed, a haircot bean seed, a lima bean seed, a butter bean seed, a adzuki bean seed, a azuki bean seed, a mung bean seed, a golden gram seed, a green gram seed, a black gram seed, a scarlet runner bean seed, a rice bean seed, a moth bean seed, and a combination thereof.

In some embodiments, the legume seed is germinating. In some embodiments, the legume seed is non-germinating. In some embodiments, (b) comprises applying a bacteria to the legume seed comprising the protein expression vector. In some embodiments, the bacteria comprises agrobacteria. In some embodiments, (b) comprises microbombardment.

In some embodiments, the method further comprises storing the legume seed at a temperature of between 0° C. and −40° C. after (c) and before (d). In some embodiments, the protein expression vector comprises an intron, an exon, or a combination thereof. In some embodiments, the protein expression vector comprises a promoter. In some embodiments, the promoter comprises a nucleotide sequence with at least 80% sequence identity to SEQ ID NO:1. In some embodiments, the protein expression vector comprises a domain encoding a signaling peptide. In some embodiments, the domain encoding a signaling peptide comprises a nucleotide sequence with at least 80% sequence identity to SEQ ID NO:2. In some embodiments, the domain encoding a signaling peptide comprises a nucleotide sequence with at least 80% sequence identity to SEQ ID NO:4. In some embodiments, the domain encoding a signaling peptide comprises a nucleotide sequence with at least 80% sequence identity to SEQ ID NO:6. In some embodiments, the domain encoding a signaling peptide comprises a nucleotide sequence with at least 80% sequence identity to SEQ ID NO:8.

In some embodiments, the protein expression vector comprises a nucleotide sequence encoding a transgene. In some embodiments, the nucleotide sequence has at least 80% sequence identity to SEQ ID NO:10. In some embodiments, the nucleotide sequence has at least 80% sequence identity to SEQ ID NO:12. In some embodiments, the nucleotide sequence has at least 80% sequence identity to SEQ ID NO:14. In some embodiments, the protein expression vector comprises a nucleotide sequence encoding a NOS terminator. In some embodiments, the NOS terminator comprises a nucleotide sequence with at least 80% sequence identity to SEQ ID NO:17. In some embodiments, the protein expression vector comprises a nucleotide sequence encoding a His-tag. In some embodiments, the His-tag comprises a nucleotide sequence with at least 80% sequence identity to SEQ ID NO:18. In some embodiments, the protein expression vector comprises a nucleotide sequence with at least 80% sequence identify to SEQ ID NO:16.

In some embodiments, the protein comprises an antibody, an enzyme, a structural protein, a signaling protein, a dairy protein, or a combination thereof. In some embodiments, the protein comprises a dairy protein and where the dairy protein is a casein protein. In some embodiments, the casein protein comprises αs1-casein, αs2-casein, β-casein, κ-casein, or a combination thereof. In some embodiments, the protein comprises an amino acid sequence with at least 80% sequence identity to SEQ ID NO:11. In some embodiments, the protein comprises an amino acid sequence with at least 80% sequence identity to SEQ ID NO:13. In some embodiments, the protein comprises an amino acid sequence with at least 80% sequence identity to SEQ ID NO:15. In some embodiments, the comprises insulin.

In some embodiments, the protein comprises a signaling peptide. In some embodiments, the signaling peptide comprises an amino acid sequence with at least 80% sequence identity to SEQ ID NO:3. In some embodiments, the signaling peptide comprises an amino acid sequence with at least 80% sequence identity to SEQ ID NO:5. In some embodiments, the signaling peptide comprises an amino acid sequence with at least 80% sequence identity to SEQ ID NO:7. In some embodiments, the signaling peptide comprises an amino acid sequence with at least 80% sequence identity to SEQ ID NO:9.

In some embodiments, the method further comprising, in (d), separating the protein from the legume seed using alkaline conditions, using acidic conditions, or a combination thereof in series. In some embodiments, the method further comprising, in (d), separating the protein from other proteins in the legume seed. In some embodiments, separating the protein comprises his-tag purification, ion-exchange chromatography, isoelectric precipitation, or a combination thereof. In some embodiments, separating the protein comprises his-tag purification and the his-tag purification comprises Immobilized Metal Affinity Chromatography (IMAC).

In another aspect, the present disclosure provides a method for generating a protein product, the method comprising: (a) providing a plant seed; (b) contacting the plant seed with a protein expression vector that encodes for a protein; (c) transiently expressing the protein in the plant seed using the protein expression vector; and (d) isolating the protein from the plant seed to obtain a protein product, where a yield of the protein product is at least about 0.1 g protein/kg seed in no more than 10 days.

In some embodiments, the yield of the protein product is at least about 0.1 g protein/kg seed in no more than 6 days. In some embodiments, the yield of the protein product is at least about 0.1 g protein/kg seed in no more than 4 days. In some embodiments, the yield of the protein product is at least about 0.1 g protein/kg seed in no more than 2 days. In some embodiments, the yield of the protein product is at least about 1 g protein/kg seed in no more than 10 days. In some embodiments, the yield of the protein product is at least about 3 g protein/kg seed in no more than 10 days. In some embodiments, the yield of the protein product is at least about 6 g protein/kg seed in no more than 10 days.

In another aspect, the present disclosure provides a method for generating a protein product, the method comprising: (a) providing a plant seed; (b) contacting the plant seed with a protein expression vector that encodes for a protein; (c) transiently expressing the protein in the plant seed using the protein expression vector; and (d) isolating the protein from the plant seed prior to a post-germination phase to obtain a protein product, where yield of the protein product is at least about 1 g protein/kg seed.

In some embodiments, the yield of the protein product is at least about 3 g protein/kg seed. In some embodiments, the yield of the protein product is at least about 6 g protein/kg seed. In some embodiments, the yield of the protein product is at least about 8 g protein/kg seed.

In some embodiments, the plant seed belongs to a plant family selected from the group consisting of Fabaceae, Poaceae, and Brassicaceae. In some embodiments, the plant seed belongs to the Fabaceae family. In some embodiments, the plant seed that belongs to the Fabaceae family is selected from the group consisting of a dry bean seed, a dry broad bean seed, a dry pea seed, a chickpea seed, a dry cowpea seed, a pigeon pea seed, a lentil seed, a bambara groundnut seed, a vetch seed, a lupin seed, a pulse NES seed, and a combination thereof.

In some embodiments, the plant seed belongs to the Fabaceae family is a dry bean. In some embodiments, the dry bean is selected from the group consisting of a kidney bean seed, a navy bean seed, a pinto bean seed, a black turple bean seed, a haircot bean seed, a lima bean seed, a butter bean seed, a adzuki bean seed, a azuki bean seed, a mung bean seed, a golden gram seed, a green gram seed, a black gram seed, a scarlet runner bean seed, a rice bean seed, a moth bean seed, and a combination thereof. In some embodiments, the plant seed belongs to the Poaceae family. In some embodiments, the plant that belongs to the Poaceae family is selected from the group consisting of a maize seed, a rice seed, a wheat seed, a barley seed, a sorghum seed, a millet seed, an oat seed, a triticale seed, a rye seed, and a fonio seed. In some embodiments, the plant seed belongs to the Brassicaceae family. In some embodiments, the plant seed that belongs to the Brassicaceae family is selected from the group consisting of a Brassica oleracea seed, a Brassica rapa seed, a Brassica napus seed, a Raphanus sativus seed, an Armoracia rusticana seed and an Arabidopsis thaliana seed.

In some embodiments, the seed is germinating. In some embodiments, the seed is non-germinating. In some embodiments, contacting the plant seed comprises applying a bacteria to the plant seed comprising the protein expression vector. In some embodiments, the bacteria comprises agrobacteria. In some embodiments, contacting the plant seed comprises microbombardment. In some embodiments, the method further comprises storing the legume seed at a temperature of between 0° C. and −40° C. after (c) and before (d).

In some embodiments, the protein expression vector comprises an intron, an exon, or a combination thereof. In some embodiments, the protein expression vector comprises a promoter. In some embodiments, the promoter comprises a nucleotide sequence with at least 80% sequence identity to SEQ ID NO:1. In some embodiments, the protein expression vector comprises a domain encoding a signaling peptide. In some embodiments, the domain encoding a signaling peptide comprises a nucleotide sequence with at least 80% sequence identity to SEQ ID NO:2. In some embodiments, the domain encoding a signaling peptide comprises a nucleotide sequence with at least 80% sequence identity to SEQ ID NO:4.

In some embodiments, the domain encoding a signaling peptide comprises a nucleotide sequence with at least 80% sequence identity to SEQ ID NO:6. In some embodiments, the domain encoding a signaling peptide comprises a nucleotide sequence with at least 80% sequence identity to SEQ ID NO:8. In some embodiments, the protein expression vector comprises a nucleotide sequence encoding a transgene. In some embodiments, the nucleotide sequence has at least 80% sequence identity to SEQ ID NO:10. In some embodiments, the nucleotide sequence has at least 80% sequence identity to SEQ ID NO:12. In some embodiments, the nucleotide sequence has at least 80% sequence identity to SEQ ID NO:14.

In some embodiments, the protein expression vector comprises a nucleotide sequence encoding a NOS terminator. In some embodiments, the NOS terminator comprises a nucleotide sequence with at least 80% sequence identity to SEQ ID NO:17. In some embodiments, the protein expression vector comprises a nucleotide sequence encoding a His-tag. In some embodiments, the His-tag comprises a nucleotide sequence with at least 80% sequence identity to SEQ ID NO:18. In some embodiments, the protein expression vector comprises a nucleotide sequence with at least 80% sequence identify to SEQ ID NO:16.

In some embodiments, the protein comprises an antibody, an enzyme, a structural protein, a signaling protein, a dairy protein, or a combination thereof. In some embodiments, the protein comprises a dairy protein and where the dairy protein is a casein protein. In some embodiments, the casein protein comprises αs1-casein, αs2-casein, β-casein, κ-casein, or a combination thereof. In some embodiments, the protein comprises an amino acid sequence with at least 80% sequence identity to SEQ ID NO:11. In some embodiments, the protein comprises an amino acid sequence with at least 80% sequence identity to SEQ ID NO:13. In some embodiments, the protein comprises an amino acid sequence with at least 80% sequence identity to SEQ ID NO:15. In some embodiments, the protein comprises insulin.

In some embodiments, the protein comprises a signaling peptide. In some embodiments, the signaling peptide comprises an amino acid sequence with at least 80% sequence identity to SEQ ID NO:3. In some embodiments, the signaling peptide comprises an amino acid sequence with at least 80% sequence identity to SEQ ID NO:5. In some embodiments, the signaling peptide comprises an amino acid sequence with at least 80% sequence identity to SEQ ID NO:7. In some embodiments, the signaling peptide comprises an amino acid sequence with at least 80% sequence identity to SEQ ID NO:9.

In some embodiments, the method further comprises, in (d), separating the protein from the plant seed using alkaline conditions, using acidic conditions, or a combination thereof in series. In some embodiments, the method further comprises, in (d), separating the protein from other proteins in the plant seed. In some embodiments, separating the protein may comprise his-tag purification, ion-exchange chromatography, isoelectric precipitation, or a combination thereof. In some embodiments, separating the protein may comprise his-tag purification and the his-tag purification comprises IMAC.

In another aspect, the present disclosure provides a method for generating a dairy protein product, the method comprising: (a) providing a plant seed; (b) contacting the plant seed with a protein expression vector that encodes for a dairy protein; (c) transiently expressing the dairy protein in the plant seed using the protein expression vector; and (d) isolating the dairy protein from the plant seed to obtain a dairy protein product.

In some embodiments, the plant seed belongs to a plant family selected from the group consisting of Fabaceae, Poaceae, and Brassicaceae. In some embodiments, the plant seed belongs to the Fabaceae family. In some embodiments, the plant seed that belongs to the Fabacae family is selected from the group consisting of a dry bean seed, a dry broad bean seed, a dry pea seed, a chickpea seed, a dry cowpea seed, a pigeon pea seed, a lentil seed, a bambara groundnut seed, a vetch seed, a lupin seed, a pulse NES seed, and a combination thereof.

In some embodiments, the plant seed belongs to the Fabaceae family is a dry bean. In some embodiments, the dry bean is selected from the group consisting of a kidney bean seed, a navy bean seed, a pinto bean seed, a black turple bean seed, a haircot bean seed, a lima bean seed, a butter bean seed, a adzuki bean seed, a azuki bean seed, a mung bean seed, a golden gram seed, a green gram seed, a black gram seed, a scarlet runner bean seed, a rice bean seed, a moth bean seed, and a combination thereof. In some embodiments, the plant belongs to the Poaceae family. In some embodiments, the plant that belongs to the Poaceae family is selected from the group consisting of a maize seed, a rice seed, a wheat seed, a barley seed, a sorghum seed, a millet seed, an oat seed, a tritcale seed, a rye seed, and a fonio seed. In some embodiments, the plant belongs to the Brassicaceae family. In some embodiments, the plant seed that belongs to the Brassicaceae family is selected from the group consisting of a Brassica oleracea seed, a Brassica rapa seed, a Brassica napus seed, a Raphanus sativus seed, an Armoracia rusticana seed and an Arabidopsis thaliana seed.

In some embodiments, the seed is germinating. In some embodiments, the seed is non-germinating. In some embodiments, contacting the plant seed comprises applying a bacteria to the plant seed comprising the dairy protein expression vector.

In some embodiments, the bacteria comprises agrobacteria. In some embodiments, contacting the plant seed comprises microbombardment. In some embodiments, the method further comprises storing the legume seed at a temperature of between 0° C. and −40° C. after (c) and before (d).

In some embodiments, the protein expression vector comprises an intron, an exon, or a combination thereof. In some embodiments, the protein expression vector comprises a promoter. In some embodiments, the promoter comprises a nucleotide sequence with at least 80% sequence identity to SEQ ID NO:1. In some embodiments, the protein expression vector comprises a domain encoding a signaling peptide. In some embodiments, the domain encoding a signaling peptide comprises a nucleotide sequence with at least 80% sequence identity to SEQ ID NO:2. In some embodiments, the domain encoding a signaling peptide comprises a nucleotide sequence with at least 80% sequence identity to SEQ ID NO:4. In some embodiments, the domain encoding a signaling peptide comprises a nucleotide sequence with at least 80% sequence identity to SEQ ID NO:6. In some embodiments, the domain encoding a signaling peptide comprises a nucleotide sequence with at least 80% sequence identity to SEQ ID NO:8.

In some embodiments, the protein expression vector comprises a nucleotide sequence encoding a transgene. In some embodiments, the nucleotide sequence has at least 80% sequence identity to SEQ ID NO:10. In some embodiments, the nucleotide sequence has at least 80% sequence identity to SEQ ID NO:12. In some embodiments, the nucleotide sequence has at least 80% sequence identity to SEQ ID NO:14. In some embodiments, the protein expression vector comprises a nucleotide sequence encoding a NOS terminator. In some embodiments, the NOS terminator comprises a nucleotide sequence with at least 80% sequence identity to SEQ ID NO:17.

In some embodiments, the protein expression vector comprises a nucleotide sequence encoding a His-tag. In some embodiments, the His-tag comprises a nucleotide sequence with at least 80% sequence identity to SEQ ID NO:18. In some embodiments, the protein expression vector comprises a nucleotide sequence with at least 80% sequence identify to SEQ ID NO:16.

In some embodiments, the dairy protein comprises a casein protein. In some embodiments, the casein protein comprises αs1-casein, αs2-casein, β-casein, κ-casein, or a combination thereof. In some embodiments, the dairy protein comprises an amino acid sequence with at least 80% sequence identity to SEQ ID NO:11. In some embodiments, the dairy protein comprises an amino acid sequence with at least 80% sequence identity to SEQ ID NO:13. In some embodiments, the dairy protein comprises an amino acid sequence with at least 80% sequence identity to SEQ ID NO:15.

In some embodiments, the dairy protein comprises a signaling peptide. In some embodiments, the signaling peptide comprises an amino acid sequence with at least 80% sequence identity to SEQ ID NO:3. In some embodiments, the signaling peptide comprises an amino acid sequence with at least 80% sequence identity to SEQ ID NO:5. In some embodiments, the signaling peptide comprises an amino acid sequence with at least 80% sequence identity to SEQ ID NO:7. In some embodiments, the signaling peptide comprises an amino acid sequence with at least 80% sequence identity to SEQ ID NO:9.

In some embodiments, separating the dairy protein may comprise extraction using alkaline conditions, extraction using acidic conditions, or a combination thereof. In some embodiments, separating the dairy protein may comprise separating the dairy protein from other proteins. In some embodiments, separating the dairy protein may comprise his-tag purification, ion-exchange chromatography, isoelectric precipitation, or a combination thereof. In some embodiments, separating the dairy protein may comprise his-tag purification, ion-exchange chromatography, isoelectric precipitation, or a combination thereof. In some embodiments, separating the dairy protein may comprise his-tag purification and the his-tag purification comprises IMAC.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a schematic conceptually depicting an example method(s) described herein.

FIG. 2 shows a schematic conceptually depicting an example method(s) described herein.

FIG. 3 shows a schematic conceptually depicting an example method(s) described herein.

FIG. 4 shows a schematic conceptually depicting an example method(s) described herein.

FIG. 5 shows a graph depicting the germination and post germination phases of seed growth.

FIG. 6 shows an SDS-PAGE gel of example expressed proteins.

FIG. 7 shows a yield chart of example expressed proteins.

FIG. 8 shows an SDS-PAGE gel of example expressed proteins.

FIG. 9 shows an SDS-PAGE gel of example expressed proteins.

FIG. 10 shows an SDS-PAGE gel of example expressed proteins.

FIG. 11 shows an SDS-PAGE gel of example His-tag purified proteins.

FIG. 12 shows a yield chart of example expressed proteins.

FIG. 13 shows mass spectrometry results for an example protein sample produced in seeds.

FIG. 14 shows pictures of water and oil mixtures with and without the casein protein produced by seeds, as described herein.

FIG. 15 shows a picture of micelles observed with the addition of kappa-casein.

FIG. 16 shows an example vegan mozzarella cheese recipe.

FIG. 17 shows a picture of coagulated cheese using kappa casein.

FIG. 18 shows an example vegan cheese recipe.

FIG. 19 shows a picture of vegan cheese made using kappa-casein powder and a negative control.

FIG. 20 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Provided herein are methods and compositions useful for the production of proteins by transient expression of proteins in plant seeds. Compared to recombinant protein expression using stable transgenic lines, transient expression is advantageous because it is faster and less resource-intensive. Plant seeds, as described herein, can accumulate high levels protein storage, which may serve as a key benefit for protein production in seeds. The methods and compositions described herein may relate to a variety of plant seed types as well as the production of a variety of proteins.

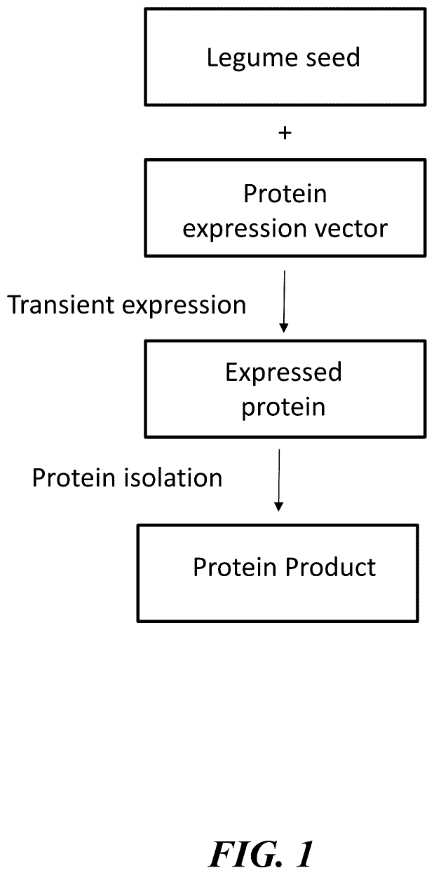

An aspect of the disclosure provides a method for generating a protein product. The method, depicted in FIG. 1, comprises: a) providing a legume seed; b) contacting the legume seed with a protein expression vector that encodes for a protein; c) transiently expressing the protein in the legume seed using the protein expression vector; and d) isolating the protein from the legume seed to obtain a protein product.

In some examples, the legume seed may be a dry bean seed, a dry broad bean seed, a dry pea seed, a chickpea seed, a dry cowpea seed, a pigeon pea seed, a lentil seed, a bambara groundnut seed, a vetch seed, a lupin seed, a pulse NES seed, or combination thereof. In some embodiments, the dry bean seed may be a kidney bean seed, a navy bean seed, a pinto bean seed, a black turple bean seed, a haircot bean seed, a lima bean seed, a butter bean seed, a adzuki bean seed, a azuki bean seed, a mung bean seed, a golden gram seed, a green gram seed, a black gram seed, a scarlet runner bean seed, a rice bean seed, a moth bean seed, or a combination thereof.

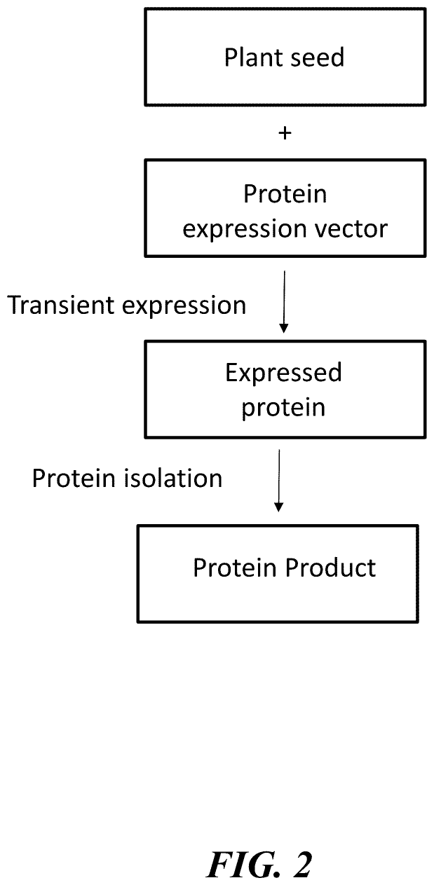

Another aspect of the disclosure provides a method for generating a protein product. The method, depicted in FIG. 2, comprises: a) providing a plant seed; b) contacting the plant seed with a protein expression vector that encodes for a protein; c) transiently expressing the protein in the plant seed using the protein expression vector; and d) isolating the protein from the plant seed to obtain a protein product, wherein yield of the protein product is at least about 1.60 g protein/kg seed in no more than 6 days.

In some cases, the yield of the protein product may be at least about 0.1 g/kg seed, at least about 0.2 g/kg seed, at least about 0.3 g/kg seed, at least about 0.4 g/kg seed, at least about 0.5 g/kg seed, at least about 0.6 g/kg seed, at least about 0.7 g/kg seed, at least about 0.7 g/kg seed, at least about 0.8 g/kg seed, at least about 0.9 g/kg seed, at least about 0.9 g/kg seed, at least about 1 g/kg seed, at least about 1.1 g/kg seed, at least about 1.1 g/kg seed, at least about 1.2 g/kg seed, at least about 1.3 g/kg seed, at least about 1.3 g/kg seed, at least about 1.4 g/kg seed, at least about 1.5 g/kg seed, at least about 1.5 g/kg seed, at least about 1.6 g/kg seed, at least about 1.7 g/kg seed, at least about 1.7 g/kg seed, at least about 1.8 g/kg seed, at least about 1.9 g/kg seed, at least about 2 g/kg seed, at least about 2.5 g/kg seed, at least about 3 g/kg seed, at least about 3.5 g/kg seed, at least about 4 g/kg seed, at least about 4.5 g/kg seed, at least about 5 g/kg seed, at least about 5.5 g/kg seed, at least about 6 g/kg seed, at least about 6.5 g/kg seed, at least about 7 g/kg seed, at least about 7.5 g/kg seed, at least about 8 g/kg seed, at least about 8.5 g/kg seed, at least about 9 g/kg seed, at least about 9.5 g/kg seed, at least about 10 g/kg seed, at least about 12 g/kg seed, at least about 14 g/kg seed, at least about 16 g/kg seed, at least about 18 g/kg seed, at least about 20 g/kg seed, at least about 25 g/kg seed, or at least about 30 g/kg seed. In some cases, the protein product yield is obtained in at most about 30 days, in at most about 29 days, in at most about 28 days, in at most about 27 days, in at most about 26 days, in at most about 25 days, in at most about 24 days, in at most about 23 days, in at most about 22 days, in at most about 21 days, in at most about 20 days, in at most about 19 days, in at most about 18 days, in at most about 17 days, in at most about 16 days, in at most about 15 days, at most about 14 days, at most about 13 days, at most about 12 days, at most about 11 days, at most about 10 days, at most about 9 days, at most about 8 days, at most about 7 days, at most about 6 days, at most about 5 days, at most about 4 days, at most about 3 days, at most about 2 days, or at most about 1 day.

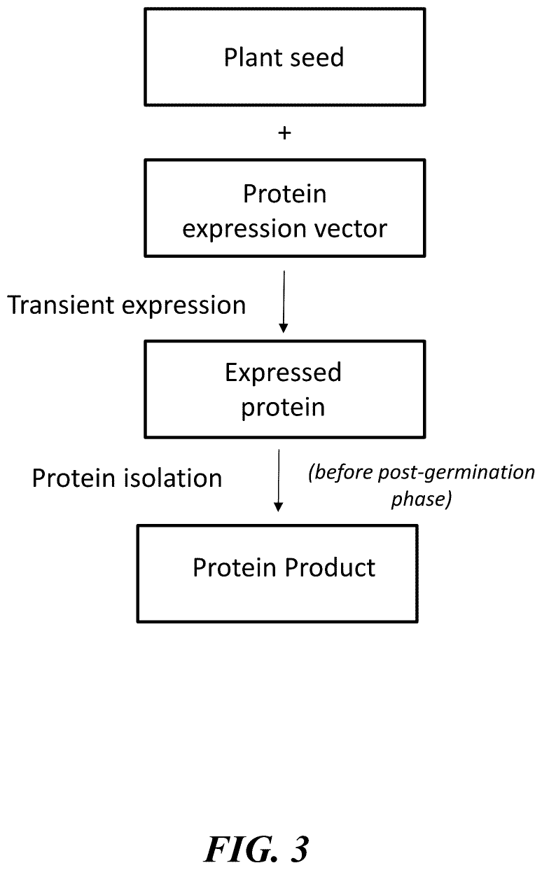

Additionally, another aspect of the disclosure provides a method for generating a protein product. The method, depicted in FIG. 3, comprises: a) providing a plant seed; b) contacting the plant seed with a protein expression vector that encodes for a protein; c) transiently expressing the protein in the plant seed using the protein expression vector; and d) isolating the protein from the plant seed prior to a post-germination phase to obtain a protein product, wherein yield of the protein product is at least about 1.6 g protein/kg seed

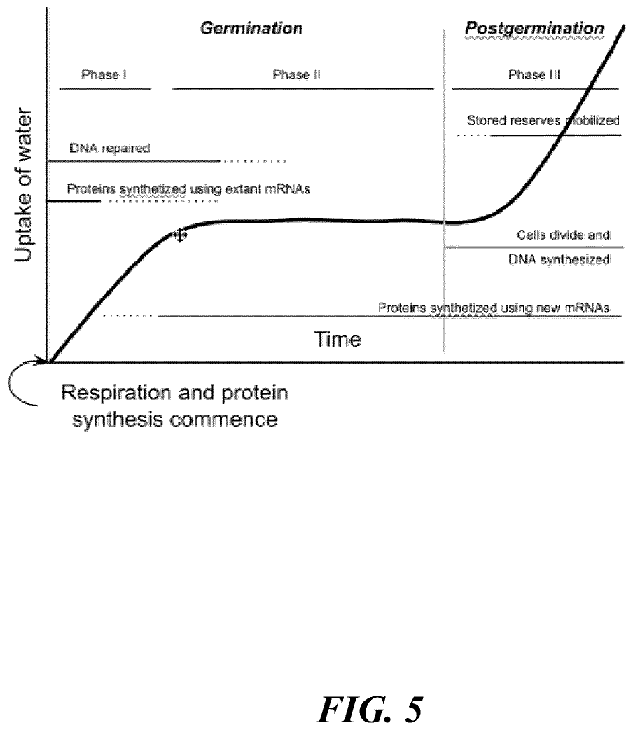

A plant seed (e.g., legume seed) may go through multiple phases of germination. There are multiple phases of seed germination in the seed plant life cycle including: phase I, rapid water imbibition by seed; phase II, reactivation of metabolism; and phase III, radicle protrusion, or post-germination phase (FIG. 5). During phase II, the essential physiological and biochemical processes such as hydrolysis, biosynthesis and mobilization of macromolecules, respiration, differentiation of subcellular structures, and cell elongation may be reactivated, resulting in initiation of germination. The post-germination phase may refer to a variety of stages after germination.

In some cases, the plant seed (e.g., legume seed) described herein may be germinating. In some cases, the plant seed (e.g., legume seed) described herein may not be germinating. The plant seed (e.g., legume seed) may be in Phase I, Phase II, Phase III, before Phase I, or after Phase III. In some cases, the plant seed (e.g., legume seed) may be in one phase at the beginning of the methods described herein and at a different phase during the methods described herein. In some cases, c) is performed prior to or during post-germination. In some cases, c) is performed after post-germination. In some cases, c) is performed during germination.

The post-germination phase may refer to a variety of stages after germination. In some embodiments, post-germination may refer to the phase of seed growth when cell division takes place. In some embodiments, post-germination may refer to the period of growth when stored nutrient reserves are consumed. In other examples, post-germination may refer to the phase when DNA is synthesized within the plant seeds. The post-germination phase may comprise protein synthesis using newly generated mRNA. In some cases, the post-germination phase may comprise a greater consumption of water by the plant seed relative to the germination phase.

In some cases, the yield of the protein product may be at least about 0.1 g/kg seed, at least about 0.2 g/kg seed, at least about 0.3 g/kg seed, at least about 0.4 g/kg seed, at least about 0.5 g/kg seed, at least about 0.6 g/kg seed, at least about 0.7 g/kg seed, at least about 0.7 g/kg seed, at least about 0.8 g/kg seed, at least about 0.9 g/kg seed, at least about 0.9 g/kg seed, at least about 1 g/kg seed, at least about 1.1 g/kg seed, at least about 1.1 g/kg seed, at least about 1.2 g/kg seed, at least about 1.3 g/kg seed, at least about 1.3 g/kg seed, at least about 1.4 g/kg seed, at least about 1.5 g/kg seed, at least about 1.5 g/kg seed, at least about 1.6 g/kg seed, at least about 1.7 g/kg seed, at least about 1.7 g/kg seed, at least about 1.8 g/kg seed, at least about 1.9 g/kg seed, at least about 2 g/kg seed, at least about 2.5 g/kg seed, at least about 3 g/kg seed, at least about 3.5 g/kg seed, at least about 4 g/kg seed, at least about 4.5 g/kg seed, at least about 5 g/kg seed, at least about 5.5 g/kg seed, at least about 6 g/kg seed, at least about 6.5 g/kg seed, at least about 7 g/kg seed, at least about 7.5 g/kg seed, at least about 8 g/kg seed, at least about 8.5 g/kg seed, at least about 9 g/kg seed, at least about 9.5 g/kg seed, at least about 10 g/kg seed, at least about 12 g/kg seed, at least about 14 g/kg seed, at least about 16 g/kg seed, at least about 18 g/kg seed, at least about 20 g/kg seed, at least about 25 g/kg seed, or at least about 30 g/kg seed.

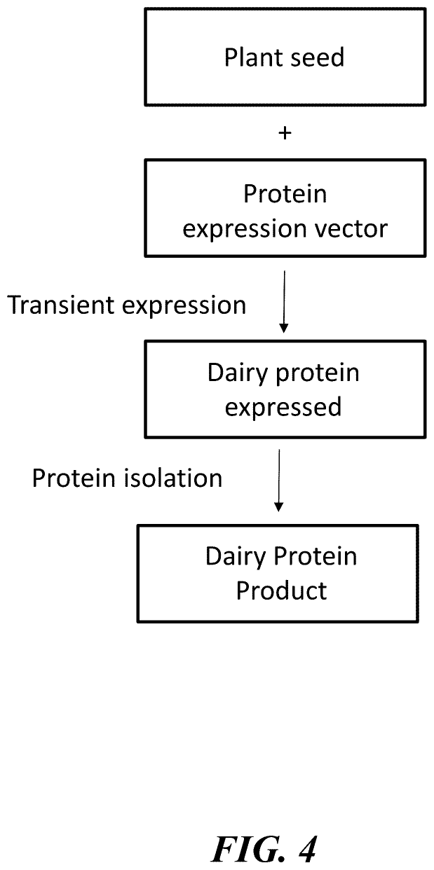

In yet another aspect of the disclosure provides a method for generating a protein product. The method, depicted in FIG. 4 comprises: a) providing a plant seed; b) contacting the plant seed with a protein expression vector that encodes for a dairy protein; c) transiently expressing the dairy protein in the plant seed using the protein expression vector; and d) isolating the dairy protein from the plant seed to obtain a dairy protein product.

In some cases, the methods described herein further comprises in d), separating the dairy protein from the plant seed (e.g., legume seed) using alkaline conditions, using acidic conditions, or a combination thereof in series. Separating the dairy protein may comprise homogenizing the plant seed (e.g., legume seed) after dairy protein expression using a blender or similar device. The homogenized plant seed may be combined with an alkaline solution and further processed. The alkaline solution may comprise a base, including but not limited to NaOH, KOH, or a combination thereof. The concentration of the base in solution may be about 0.01-5N, about 0.1-4N, about 0.2-3.8N, about 0.3-3.6N, about 0.4-3.4N, about 0.5-3.2N, about 0.6-3.2N, about 0.7-3.0N, about 0.8-2.8N, about 0.9-2.6N, about 1.0-2.4N, about 1.2-2.2N, about 1.4-2.0N, or about 1.6-1.8N. The homogenized plant seed and alkaline solution may be mixed, agitated, centrifuged, or a combination thereof. The supernatant of the homogenized plant seed and alkaline solution may be extracted and may contain the dairy protein. In some embodiments, the supernatant may be further processed using an acid solution, including but not limited to acetic acid, o-phosphoric acid, hydrochloric acid, sulfuric acid, or a combination thereof.

In some cases, the dairy protein expressed using the methods described herein may be extracted from other dairy proteins of the plant seed. The extraction may be performed using a variety of techniques, including but not limited to size-exclusion chromatography, electrophoresis, high-performance liquid chromatography, sonication, salting out, elution, filtration, or a combination thereof. The extraction may be performed based on one or a variety of features of the dairy protein, including but not limited to the dairy protein size, shape, molecular weight, isoelectric point, modification status, sequence, or a combination thereof. In some aspects, the dairy protein may comprise a His-tag and the His-tag may be used to purify the dairy protein. An antibody which recognizes the His-tag may be used to bind to the His-tag and thereby bind to the dairy protein. The antibody which recognizes the His-tag may be configured within a column, on a resin, on a solid support, or a combination thereof. The dairy protein may be extracted by binding of the His-tag connected to the dairy protein to the antibody and washing the column, resin, or solid support to remove other components. The dairy protein may be eluted from the antibody using one or a variety of methods including heat, salt, agitation, pH, solvents, or a combination thereof.

There are multiple phases of seed germination in the seed plant life cycle including: phase I, rapid water imbibition by seed; phase II, reactivation of metabolism; and phase III, radicle protrusion, or post-germination phase. During phase II, the essential physiological and biochemical processes such as hydrolysis, biosynthesis and mobilization of macromolecules, respiration, differentiation of subcellular structures, and cell elongation may be reactivated, resulting in initiation of germination. The post-germination phase may refer to a variety of stages after germination.

In some aspects, the plant seed (e.g., legume seed) described herein may be germinating. In some aspects, the plant seed (e.g., legume seed) described herein may not be germinating. The plant seed may be in Phase I, Phase II, Phase III, before Phase I, or after Phase III. In some cases, the plant seed may be in one phase at the beginning of the methods described herein and at a different phase during the methods described herein.

In some aspects, the plant seed (e.g., legume seed) may belong to a variety of families, including but not limited to the Fabaceae family, the Poaceae family, the Brassicaceae family, or a combination thereof. In some cases, the plat seed may belong to the Fabaceae family and may comprise a dry bean seed, a dry broad bean seed, a dry pea seed, a chickpea seed, a dry cowpea seed, a pigeon pea seed, a lentil seed, a bambara groundnut seed, a vetch seed, a lupin seed, a pulse NES seed, or a combination thereof. In some cases, the plant seed may be a dry bean and may comprise a kidney bean seed, a navy bean seed, a pinto bean seed, a black turple bean seed, a haircot bean seed, a lima bean seed, a butter bean seed, a adzuki bean seed, a azuki bean seed, a mung bean seed, a golden gram seed, a green gram seed, a black gram seed, a scarlet runner bean seed, a rice bean seed, a moth bean seed, or a combination thereof. In some cases, the plant seed may belong to the Poaceae family and may comprise a maize seed, a rice seed, a wheat seed, a barley seed, a sorghum seed, a millet seed, an oat seed, a triticale seed, a rye seed, a fonio seed, or a combination thereof. In some cases, the plant seed may belong to the Brassicaceae family and may comprise a Brassica oleracea seed, a Brassica rapa seed, a Brassica napus seed, a Raphanus sativus seed, an Armoracia rusticana seed and an Arabidopsis thaliana seed.

In some aspects, b) may comprise applying a bacteria comprising the protein expression vector to the plant seed (e.g., legume seed). The bacteria comprising the protein expression vector may be co-incubated with the plant seed (e.g., legume seed). In some cases, the bacteria comprising the protein expression vector may be co-incubated with the plant seed at a given temperature. The given temperature may be about 4-45° C., about 8-40° C., about 10-35° C., about 15-30° C., about 18-28° C., or about 23-25° C. In some cases, the bacteria comprising the protein expression vector and the plant seed may be agitated. The agitation may cause transfer of the protein expression vector from the bacteria to the plant seed.

In some aspects, the bacteria comprising the protein expression vector may be prepared using a heat-shock method. The heat-shock method may comprise subjecting the bacteria to a short duration of heat, which may induce the formation of pores. In some aspects, the bacteria comprising the protein expression vector may be prepared using electroporation. In some embodiments, the electroporation may comprise the use of high-voltage pulses. The high-voltage pulses may cause reversible breakdown of the cell membrane. In some embodiments, the protein expression vector may enter the bacteria cell through the pores formed by heat shock, electroporation, or a combination thereof. The bacteria cells may be plated and incubated after heat shock, electroporation, or a combination thereof. The plated bacteria may form colonies after incubation, wherein the colonies that grow may represent the bacteria cells containing the vector introduced during the heat shock process.

In some aspects, the bacteria may be suspended in liquid media. The liquid media may comprise a variety of components, including but not limited to media, salts, nutrients, additives, or a combination thereof. In some cases, the bacteria suspended in liquid media may be co-incubated with the plant seed (e.g., legume seed), as described herein. In some cases, more than one volume of bacteria suspended in liquid media may be co-incubated with the plant seed (e.g., legume seed) in series. In some cases, about 1-15 volumes, about 2-14, about 3-12, about 4-11, about 5-10, about 6-9, or about 7-8 volumes of bacteria suspended in liquid media may be co-incubated with the plant seed in series. In some cases, the plant seeds (e.g., legume seed) may be washed with a buffer, additional media, or a combination thereof in between co-incubating volumes of bacteria suspended in media with the plant seed. In some cases, the bacteria and plant seed may be co-incubated in the dark. In some embodiments, the bacteria and plant seed may be co-incubated in the light.

In some aspects, the bacteria may be a gram-positive bacteria, a gram-negative bacteria, or a combination thereof. In some embodiments, the bacteria may be an agrobacteria. In some embodiments, the agrobacteria may be Agrobacterium tumefaciens.

In some aspects, b) may comprise microbombardment. Microbombardment may comprise delivering nucleic acid coated particles to the plant seed (e.g., legume seed). In some cases, microbombardment is performed using a high-velocity projectile apparatus. The high-velocity projectile apparatus may inject the nucleic acid coated particles into the plant seed, using force e.g. In some cases, the high velocity projectile apparatus may inject the nucleic acid coated particles through a screen. The injection process may comprise a gas, including but not limited to an inert gas, e.g., Helium gas. The nucleic acid coated particles may comprise at least one copy of the protein expression vector. In some cases, additional components may be delivered to the plant seed (e.g., legume seed) in combination with the nucleic acid coated particles. In some cases, the nucleic acid coated particle may comprise additional nucleic acid molecules.

In some aspects, the methods described herein may further comprise storing the plant seed at a given temperature after c) and before d). The given temperature may be about −80-80° C., about −75-75° C., about −70-70° C., about −65-65° C., about −60-60° C., about −55-55° C., about −50-50° C., about −45-45° C., about −40-40° C., about −35-35° C., about −30-30° C., about −25-25° C., about −20-20° C., about −15-15° C., about −10-10° C., or about −5-5° C. The plant seed (e.g. legume seed) may be stored in a freezer, an incubator, a cryopreservation device, refrigerator, or a combination thereof. In some cases, the plant seed may be stored at least about 4 hours, at least about 8 hours, at least about 12 hours, at least about 24 hours, at least about 1 day, at least about 2 days, at least about 3 days, at least about 4 days, at least about 5 days, at least about 1 week, at least about 2 weeks, at least about 3 weeks, at least about 4 weeks, at least about 1 month, at least about 2 months, at least about 3 months, at least about 4 months, at least about 5 months, at least about 6 months or more.

In some aspects, the protein expression vector may comprise a nucleotide sequence. The nucleotide sequence may comprise a variety of components including but not limited to a promoter, an enhancer, an intron, an exon, a transgene, a domain encoding a signaling peptide, a His-Tag, a terminator, or a combination thereof. The nucleotide sequence may be codon optimized for expression in plant seed (e.g., legume seed), including but not limited to the plant seeds described herein. In some cases, the nucleotide sequence may be codon optimized for expression in legume seeds, including but not limited to the legume seeds described herein. In some embodiments, the nucleotide sequence may encode for an amino acid sequence. The amino acid sequence may comprise a variety of components including, but not limited to a transgene, a domain encoding a signaling peptide, a His-Tag, or a combination thereof.

In some aspects, more than one protein expression vector may be used in the methods described herein. In some aspects, about 1-100, about 2-90, about 3-80, about 4-70, about 5-60, about 6-55, about 7-50, about 8-45, about 9-40, about 10-35, about 15-30, about 20-25 protein expression vectors may be used in the methods described herein. In some embodiments, the more than one protein expression vectors may comprise components that are the same. In some embodiments, the more than one protein expression vectors may comprise components that are different. In some embodiments, the more than one protein expression vector may comprise different transgenes. In some cases, the different transgenes may encode of different subunits of a protein complex. In some cases, the different transgenes may encode for different enzymes of a signaling cascade.

In some aspects, the protein expression vector may be generated using cloning techniques. In some cases, certain components of the protein expression vector may be added by insertion using restriction enzyme cut sites and a DNA ligase. In some cases, the protein expression vector may comprise a plasmid or portion thereof. In some embodiments, the plasmid or portion thereof may be based on a plant virus expression vector. In some cases, the plant virus expression vector may be based on a Tobamovirus, a Potexvirus, a Comovirus, a Geminivirus, or a combination thereof. In some cases, the protein expression vector may comprise a selection gene, comprising a resistance gene in the presence of an antibiotic, e.g., kanamycin. In some cases. The plasmid or portion thereof may comprise a pCambia0380 vector, a pCambia2300 vector, a pNosdcGUS vector, a pYPQ203 (pMDC32-Ubi1) vector, a pJL-TRBO vector, a pYPQ202 vector, a pFZ19 vector, a pFZ19 vector, a pGWB501 vector, a pGWB402 vector, a pTA7001-DEST vector, a pGWB502 vector, a pGTQL1211YN vector, or a combination thereof.

In some aspects, the protein expression vector may comprise a promoter. The promoter sequence may comprise a nucleotide sequence. The promoter sequence may comprise a CMV promoter, EF1a promoter, an SV40 promoter, a PGK1 promoter, a Ubc promoter, a human beta actin promoter, a CAG promoter, a TRE promoter, an UAS promoter, an Ac5 promoter, a Polyhedrin promoter, a caMKIIa promoter, a GALI promoter, a TEF1 promoter, a GDS promoter, an ADHI promoter, a CaMV35S promoter, or a combination thereof. In some aspects, the promoter sequence may comprise a nucleotide sequence with at least about 50%, at least about 55%, at least about 60%, at least about 65%, at least about 70%, at least about 75%, at least about 80%, at least about 85%, at least about 85%, at least about 90%, at least about 95%, at least about 99%, or at least about 100% sequence identity to SEQ ID NO:1.

In some aspects, the protein expression vector may comprise a domain encoding a signaling peptide. The domain encoding a signaling peptide may comprise a nucleotide sequence. The nucleotide sequence may encode for an amino acid sequence. The domain encoding a signaling peptide may provide certain advantages to the expression of the proteins described herein. In some cases, the advantages may be enhanced expression levels, a faster rate of expression, or a combination thereof. In some cases, the signaling peptide may be based on the sequence of a signaling peptide used an organism, e.g., a mammalian organism, a bacterial organism, a plant organism, a fungal organism, or a combination thereof. In some aspects, the domain encoding a signaling peptide may comprise a nucleotide sequence with at least about 50%, at least about 55%, at least about 60%, at least about 65%, at least about 70%, at least about 75%, at least about 80%, at least about 85%, at least about 85%, at least about 90%, at least about 95%, at least about 99%, or at least about 100% sequence identity to SEQ ID NO: 2, 4, 6, or 8.

In some aspects, the protein expression vector may comprise a transgene. The transgene may comprise a nucleotide sequence. The nucleotide sequence may encode for an amino acid sequence. The amino acid sequence may comprise the amino acid sequence of one or any combination of the proteins described herein, e.g., a dairy protein, an antibody, or insulin. In certain aspects, the transgene may comprise a sequence for a casein protein. The casein protein may comprise αs1-casein, αs2-casein, β-casein, κ-casein, or a combination thereof. In some aspects, the transgene may comprise a nucleotide sequence with at least about 50%, at least about 55%, at least about 60%, at least about 65%, at least about 70%, at least about 75%, at least about 80%, at least about 85%, at least about 85%, at least about 90%, at least about 95%, at least about 99%, or at least about 100% sequence identity to SEQ ID NO: 10, 12, 14, or 16.

In some aspects, the protein expression vector may comprise a terminator. The terminator may comprise a nucleotide sequence. The terminator may contribute to transcription inhibition. The terminator may comprise a nopaline synthase (NOS) terminator. In some aspects, the terminator may comprise a nucleotide sequence with at least about 50%, at least about 55%, at least about 60%, at least about 65%, at least about 70%, at least about 75%, at least about 80%, at least about 85%, at least about 85%, at least about 90%, at least about 95%, at least about 99%, or at least about 100% sequence identity to SEQ ID NO: 17.

In some aspects, the protein expression vector may comprise a nucleotide sequence encoding a His-tag. The His-tag may be used for purification of the protein product described herein. In some cases, the His-tag may be present on the protein product. In some cases, the His-tag may be removed from the protein product In certain aspects, the His-tag may comprise multiple histidine residues. The multiple histidine residues may comprise about 1-20, about 2-19, about 3-18, about 4-17, about 5-16, about 6-15, about 6-14, about 7-13, about 8-12, about 9-11, or about 10 histidine residues. In some aspects, the nucleotide sequence encoding a His-tag may comprise a nucleotide sequence with at least about 50%, at least about 55%, at least about 60%, at least about 65%, at least about 70%, at least about 75%, at least about 80%, at least about 85%, at least about 85%, at least about 90%, at least about 95%, at least about 99%, or at least about 100% sequence identity to SEQ ID NO: 18.

In some aspects, the protein expression vector may comprise a nucleotide sequence with at least about 50%, at least about 55%, at least about 60%, at least about 65%, at least about 70%, at least about 75%, at least about 80%, at least about 85%, at least about 85%, at least about 90%, at least about 95%, at least about 99%, or at least about 100% sequence identity to SEQ ID NO: 16.

In some aspects, the methods described herein produce a protein. The protein may comprise an antibody, an enzyme, a structural protein, a signaling protein, a dairy protein, or a combination thereof. Production of proteins within the seeds described herein has certain advantages over other methods, including but not limited to, inclusion of a diverse set of post-translational modifications (PTMs), high yields, and rapid production. The proteins described herein, may comprise certain PTMs, including but not limited to glycosylation, phosphorylation, methylation, acetylation, ubiquitination, SUMOylation, or a combination thereof. In some aspects, the protein may be a mammalian protein, a bacterial protein, a fungal protein, or a combination thereof.

In some aspects, the protein may comprise an antibody. In certain embodiments, the antibody may be used for therapeutic purposes. In some embodiments, the antibody may be used for research purposes. The antibody may recognize and bind to a target. The target may be a protein, a small molecule, a nucleic acid, a macromolecule, or a combination thereof. In some cases, the target may be a mammalian target, a bacterial target, a fungal target, or a combination thereof. In some embodiments, the target may be a cancer target. The cancer target may be a mutated target, an overexpressed target, an under expressed target, or a combination thereof. In some cases, the antibody may be a variety of isotypes, including but not limited to, IgG, IgM, IgD, IgA, or a combination thereof.

In some aspects, the protein may comprise a signaling protein. The signaling protein may be a mammalian signaling protein, a bacterial signaling protein, a fungal signaling protein, or a combination thereof. In some aspects, the signaling protein may be a cytokine, a chemokine, a receptor, or a combination thereof. In some cases, the receptor may be a cell surface receptor, an ion channel linked receptor, a G-protein coupled receptor, an enzyme linked receptor, an intracellular receptor, a steroid hormone receptor, or a combination thereof. In some aspects, the signaling peptide may comprise an amino acid sequence with at least about 50%, at least about 55%, at least about 60%, at least about 65%, at least about 70%, at least about 75%, at least about 80%, at least about 85%, at least about 85%, at least about 90%, at least about 95%, at least about 99%, or at least about 100% sequence identity to SEQ ID NO: 3, 5, 7, or 9.

In some aspects, the protein may comprise an enzyme. The enzyme may be a mammalian enzyme, bacterial enzyme, fungal enzyme, or a combination thereof. In some aspects, the enzyme may be functional. In some aspects, the enzyme may catalyze a chemical reaction. The enzyme may comprise an oxireductase, a hydrolase, an isomerase, a transferase, a lyase, an amylase, a DNA polymerase, an RNA polymerase, a cellulase, a carbonic anhydrase, a DNA ligase, a catalase, a ligase or a combination thereof.

In some aspects, the protein may comprise a dairy protein. The dairy protein may be used in food production. In some cases, the dairy protein is a mammalian protein. In certain cases, the dairy protein may be a component of cheese, milk, butter, yogurt, sour cream, cottage cheese, or a combination thereof. In some aspects, the dairy protein may be used to preserve food, manufacture food, or a combination thereof. The dairy protein may be used to emulsify components within a food mixture. In some embodiments, the dairy protein may be a casein protein, an albumin protein, a whey protein, a catalase protein, a lactoperoxidase protein, a lipase protein, a xanthine oxidase protein, an acid phosphatase protein, an amylase protein, a proteinase protein, a lactase protein, a rennet protein, a superoxide dismutase protein, or a combination thereof.

In some aspects, the dairy protein may be a casein protein. The casein protein may be phosphorylated or otherwise modified. In some aspects the casein protein may comprise one subunit. In some aspects, the casein protein may comprise more than one subunit, including but not limited to αs1-casein, αs2-casein, β-casein, κ-casein, or a combination thereof. In some cases, the casein protein may comprise a modification. In some aspects, the modification may comprise a His-tag, a phosphorylation, a methylation, a signaling peptide, or a combination thereof. In some aspects, the casein protein may be conjugated to another protein, e.g., a protein as described herein.

In some aspects, the protein may comprise an amino acid sequence with at least about 50%, at least about 55%, at least about 60%, at least about 65%, at least about 70%, at least about 75%, at least about 80%, at least about 85%, at least about 85%, at least about 90%, at least about 95%, at least about 99%, or at least about 100% sequence identity to SEQ ID NO:11, 13, or 15. In some aspects, the dairy protein may comprise an amino acid sequence with at least about 50%, at least about 55%, at least about 60%, at least about 65%, at least about 70%, at least about 75%, at least about 80%, at least about 85%, at least about 85%, at least about 90%, at least about 95%, at least about 99%, or at least about 100% sequence identity to SEQ ID NO: 11, 13, or 15.

In some aspects, the protein may comprise insulin. In some aspects, the insulin may comprise full-length insulin. In some aspects, the insulin may comprise proinsulin. In some aspects, the insulin may comprise an A chain, a B chain, or a combination thereof. In some aspects, more than one form of insulin may be expressed using the methods described herein. In some aspects, the insulin may comprise a modification, including but not limited to, a phosphorylation, a methylation, a disulfide, a His-tag, or a combination thereof.

In some aspects, the methods described herein further comprises in d), extraction the protein from the plant seed (e.g., legume seed) using a chemical extraction, physical extraction, or a combination thereof. In some embodiments, the chemical extraction may comprise using a chemical, a solvent, a salt, a buffer, or a combination thereof. In some embodiments, the chemical extraction may comprise adding a solution to the plant seed that causes the protein to precipitate. In some cases, the chemical extraction may comprise adding a solution to the plant seed that causes a component of the plant seed to precipitate. In some embodiments, the physical extraction may comprise filtering, chromatography, electrophoresis, or a combination thereof.

In some aspects, the methods described herein further comprises in d), separating the protein from the plant seed (e.g., legume seed) using alkaline conditions, using acidic conditions, or a combination thereof in series. Separating the protein may comprise homogenizing the plant seed (e.g., legume seed) after protein expression using a blender or similar device. The homogenized plant seed (e.g., legume seed) may be combined with an alkaline solution and further processed. The alkaline solution may comprise a base, including but not limited to NaOH, KOH, or a combination thereof. The concentration of the base in solution may be about 0.01-5N, about 0.1-4N, about 0.2-3.8N, about 0.3-3.6N, about 0.4-3.4N, about 0.5-3.2N, about 0.6-3.2N, about 0.7-3.0N, about 0.8-2.8N, about 0.9-2.6N, about 1.0-2.4N, about 1.2-2.2N, about 1.4-2.0N, or about 1.6-1.8N. The homogenized plant seed (e.g., legume seed) and alkaline solution may be mixed, agitated, centrifuged, or a combination thereof. The supernatant of the homogenized plant seed (e.g., legume seed) and alkaline solution may be extracted and may contain the protein. In some embodiments, the supernatant may be further processed using an acid solution, including but not limited to acetic acid, o-phosphoric acid, hydrochloric acid, sulfuric acid, or a combination thereof.

In some aspects, the protein expressed using the methods described herein may be separated from other proteins of the plant seed (e.g., legume seed). The separation process may be performed using a variety of techniques, including but not limited to size-exclusion chromatography, electrophoresis, high-performance liquid chromatography, sonication, salting out, elution, filtration, or a combination thereof. The separation may be performed based on one or a variety of features of the protein, including but not limited to the protein size, shape, molecular weight, isoelectric point, modification status, sequence, or a combination thereof. In some aspects, the protein may comprise a His-tag and the His-tag may be used to purify the protein. An antibody which recognizes the His-tag may be used to bind to the His-tag and thereby bind to the protein. The antibody which recognizes the His-tag may be configured within a column, on a resin, on a solid support, or a combination thereof. The protein may be separated by binding of the His-tag connected to the protein to the antibody and washing the column, resin, or solid support to remove other components. The protein may be eluted from the antibody using one or a variety of methods including heat, salt, agitation, pH, solvents, or a combination thereof. In some embodiments, immobilized-metal affinity chromatography (IMAC) may be used to purify the protein using the His-Tag. In some cases, IMAC is performed using denaturing conditions. In some cases, IMAC is performed using non-denaturing conditions. The IMAC may comprise a resin. The resin may comprise a transition metal ion including, but not limited to Co²⁺, Ni²⁺, Cu²⁺, Zn²⁺, or a combination thereof. The protein may bind to the IMAC resin. The protein may be eluted from the IMAC resin using heat, salt, imidazole, solvent, or a combination thereof.

### Computer Systems

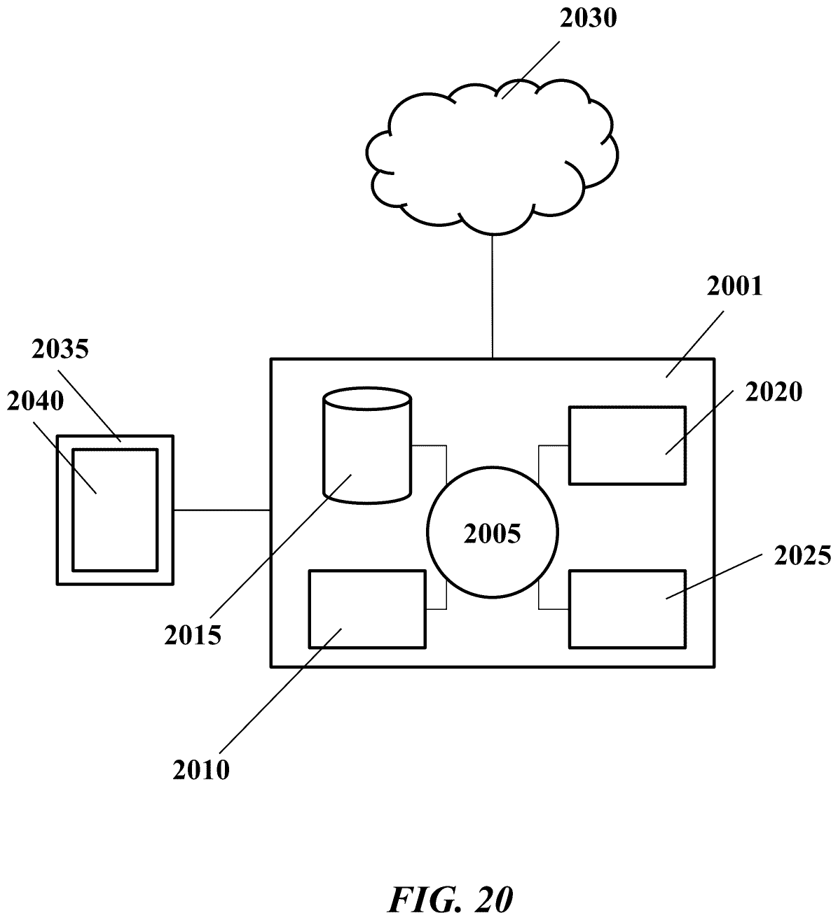

The present disclosure provides computer systems that are programmed to implement methods of the disclosure. FIG. 20 shows a computer system 2001 that is programmed or otherwise configured to control protein expression in plant seeds. The computer system 2001 can regulate various aspects of plant seed growth and protein extraction of the present disclosure, such as, for example, temperature control, humidity control, pumping speed, column pressure, exposure to light, access to water, or a combination thereof. The computer system 2001 can be an electronic device of a user or a computer system that is remotely located with respect to the electronic device. The electronic device can be a mobile electronic device.

The computer system 2001 includes a central processing unit (CPU, also “processor” and “computer processor” herein) 2005, which can be a single core or multi core processor, or a plurality of processors for parallel processing. The computer system 2001 also includes memory or memory location 2010 (e.g., random-access memory, read-only memory, flash memory), electronic storage unit 2015 (e.g., hard disk), communication interface 2020 (e.g., network adapter) for communicating with one or more other systems, and peripheral devices 2025, such as cache, other memory, data storage and/or electronic display adapters. The memory 2010, storage unit 2015, interface 2020 and peripheral devices 2025 are in communication with the CPU 2005 through a communication bus (solid lines), such as a motherboard. The storage unit 2015 can be a data storage unit (or data repository) for storing data. The computer system 2001 can be operatively coupled to a computer network (“network”) 2030 with the aid of the communication interface 2020. The network 2030 can be the Internet, an internet and/or extranet, or an intranet and/or extranet that is in communication with the Internet. The network 2030 in some cases is a telecommunication and/or data network. The network 2030 can include one or more computer servers, which can enable distributed computing, such as cloud computing. The network 2030, in some cases with the aid of the computer system 2001, can implement a peer-to-peer network, which may enable devices coupled to the computer system 2001 to behave as a client or a server.

The CPU 2005 can execute a sequence of machine-readable instructions, which can be embodied in a program or software. The instructions may be stored in a memory location, such as the memory 2010. The instructions can be directed to the CPU 2005, which can subsequently program or otherwise configure the CPU 2005 to implement methods of the present disclosure. Examples of operations performed by the CPU 2005 can include fetch, decode, execute, and writeback.

The CPU 2005 can be part of a circuit, such as an integrated circuit. One or more other components of the system 2001 can be included in the circuit. In some cases, the circuit is an application specific integrated circuit (ASIC).

The storage unit 2015 can store files, such as drivers, libraries and saved programs. The storage unit 2015 can store user data, e.g., user preferences and user programs. The computer system 2001 in some cases can include one or more additional data storage units that are external to the computer system 2001, such as located on a remote server that is in communication with the computer system 2001 through an intranet or the Internet.

The computer system 2001 can communicate with one or more remote computer systems through the network 2030. For instance, the computer system 2001 can communicate with a remote computer system of a user (e.g., an automated greenhouse). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 2001 via the network 2030.

Methods as described herein can be implemented by way of machine (e.g., computer processor) executable code stored on an electronic storage location of the computer system 2001, such as, for example, on the memory 2010 or electronic storage unit 2015. The machine executable or machine readable code can be provided in the form of software. During use, the code can be executed by the processor 2005. In some cases, the code can be retrieved from the storage unit 2015 and stored on the memory 2010 for ready access by the processor 2005. In some situations, the electronic storage unit 2015 can be precluded, and machine-executable instructions are stored on memory 2010.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 2001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2001 can include or be in communication with an electronic display 2035 that comprises a user interface (UI) 2040 for providing, for example, operating parameters and conditions, options to control the temperature, humidity level or flow rate, yield rates, process progress, etc. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2005. The algorithm can, for example, calculate values, measure variances, analyze image data, analyze tabulated data, measure minimum values, measure maximum values, analyze mass spectrometry data, calculate flow rates, or quantify humidity levels.

### EXAMPLES

### Example 1: Transient Protein Expression of GFP and TGFβ-2 in Plant Seeds

Two proteins, including green fluorescent protein (GFP), and transforming growth factor β-2 (TGFβ-2), were transiently expressed in plant seeds Protein expression vectors were designed for each protein to include a transgene, a promoter with duplicated enhanced regions, a domain encoding a signal peptide, a His-tag sequence, and a NOS terminator sequence. The transgene sequence was codon optimized in each case using the codon usage database (kazusa.jp). A construct for each protein was cloned into a pCAMBIA2300 vector using the EcoRI and BamHI restriction sites.

A sample of Agrobacterium tumefaciens was transformed using each of the expression vectors using heat shock. The transformed bacteria was then plated on an agar plate with kanamycin to select for colonies that include the expression vector. Single colonies were selected for further overnight culture. The Agrobacterium cells were harvested by centrifugation and resuspended with infiltration medium (9 mM MES, 10 mM MgCl₂, 100 μM acetosyringone). Solutions for transient expression of each protein were setup using the harvested transformed agrobacterium (200-600 mL) and plant seeds (100-300 g). A control sample was setup using a non-transformed Agrobacterium sample. The co-culture solutions were incubated in the dark for about 24 hours, after which, the seeds were rinsed and incubated again with a fresh solution of agrobacterium (200-600 mL) for about 24 hours in the dark. This process was completed again for a total of three incubations with transformed agrobacterium.

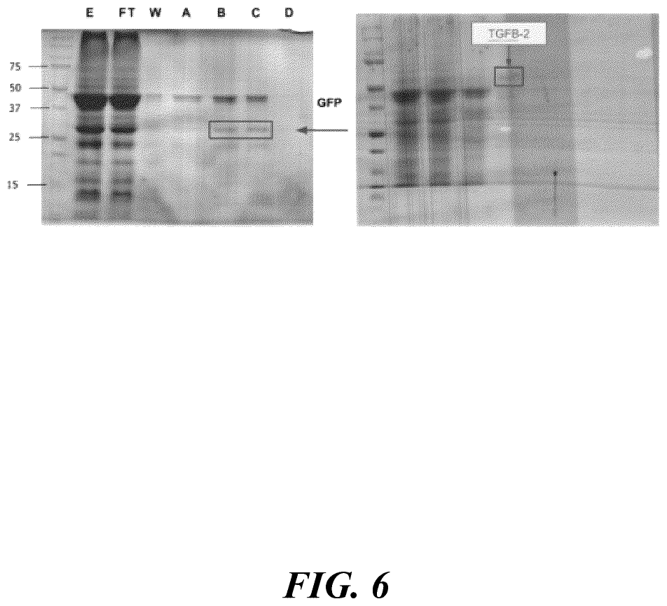

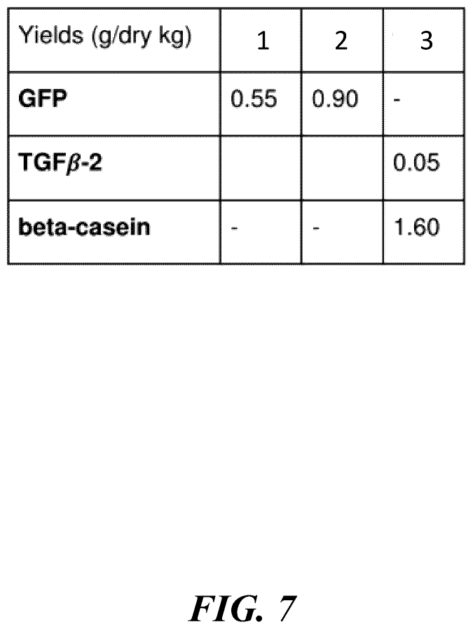

The seeds from each condition were initially isolated and stored at −20° C. to 0° C. Protein was extracted from each of the samples using alkaline processing. Briefly, 10-30 g of seeds were homogenized in a blender with 50-150 mL of 0.05-0.15 N NaOH. The samples were transferred to a tube and centrifuged at 3000-5000 rpm for 10-20 minutes. The supernatant was collected into a new tube and the pellet was discarded. His-tag purification was performed using immobilized metal affinity chromatography (IMAC), using a HisTrap FF (Cytiva) column. The column was equilibrated with 3-8 volumes of Tris-HCl 0.1 M pH 6.5-7.0. The samples were loaded onto the column and washed with 10-15 volumes of Tris-HCl 0.1M pH 6.5-7.0 and eluted with an imidazole gradient between 0-500 mM. Fractions were collected containing 0 mM, 50 mM, 100 mM and 500 mM imidazole. The fractions were analyzed using SDS PAGE electrophoresis (FIG. 6). As shown in the gel on the left, bands with the expected molecular weight of GFP were detected in fractions B and C. A band reflective of the molecular weight of TGFβ-2 was detected in the first lane of the fractions shown on the gel on the right. The yield data for production of these proteins is listed in FIG. 7. This example demonstrates that proteins like GFP and TGFβ-2 can be expressed and isolated using plant seed transient protein expression.

### Example 2: Transient Protein Expression of Beta-Casein Using Soybean, Pinto Bead and Chickpea Seeds

In this example, beta-casein was transiently expressed using three different plant seeds: soybean, pinto bean and chickpea. A protein expression vector containing a β-casein transgene was generated based on the native Bos taurus sequence (Uniprot P05814), which was optimized for expression in plants based on the codon usage database (kazusa.jp). A version of the CaMV35S promoter with duplicated enhanced region was selected for this gene construct, the KDEL signal peptide (SEQ ID NO: 19) was added to target the expression towards the secretory pathway. A 10× histidine tag (SEQ ID NO: 20) was added to the N-terminal to facilitate protein purification, and the nopaline synthase (NOS) terminator was added to the construct. The expression cassette was cloned between the sites EcoRI and BamHI of the pCAMBIA2300 vector.

Seeds: Phaseolus vulgaris cv. Pinto Saltillo seeds, Glycine max seeds and Cicer arietinum seeds were obtained at a local food market. The full, intact and healthy seeds were selected and weighed.

Bacterium: A. tumefaciens strain GV3101 was transformed with the plasmid produced as described above using heat shock. Once transformed, the culture was incubated for 48 h at 25-30° C. A single colony of A. tumefaciens strain GV3101 containing the plasmid was inoculated in 5-15 mL of LB liquid medium (25-100 μg/mL kanamycin) and was incubated during 48 hours at 25-30° C. at 200 rpm. This solution was then transferred to 300-500 mL of fresh LB medium supplemented with kanamycin (25-100 μg/ml). The culture was grown overnight at 25-30° C. at 200 rpm. The cells were harvested by centrifugation at 2000˜3000 rpm for 40-50 min at room temperature.

Co-culture: The collected cells were resuspended with infiltration medium (9 mM MES, 10 mM MgCl2, 100 μM acetosyringone) to obtain an optical density (OD) of approximately 0.5-1.5. Subsequently, 100-300 g of seeds were soaked in two volumes (100-300 mL) of the Agrobacterium culture resuspended in MES. Seeds were co-cultured with Agrobacterium for about 18-24 h at 25-30° C. in dark conditions. After co-culturing, the seeds were rinsed and incubated for an additional 18-24 hours with 1-3 volumes of fresh Agrobacterium culture resuspended in MES. In total, three consecutive infections were carried out. After the infections, the seeds were stored at −20° C. to 0° C. Control samples were incubated under the same conditions, but without Agrobacterium infection.

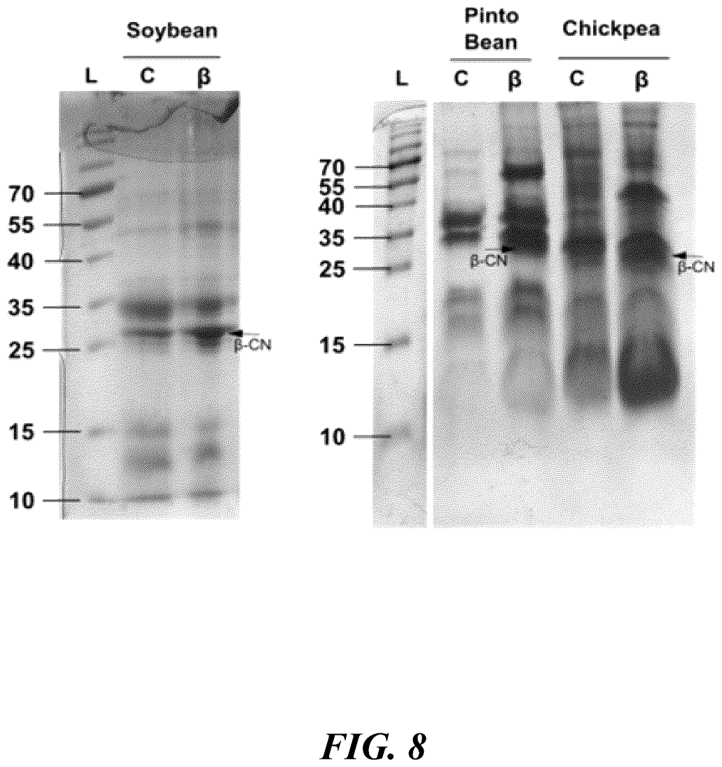

Protein from the seed samples was extracted under alkaline conditions. Briefly, 15-25 g of each seed sample were homogenized in a blender with 100 mL of NaOH 0.1 N, which represents a dilution of 1:5 (v/v). Then, the samples were transferred to 10-20 ml tubes and centrifuged at 3000-5000 rpm for 10-20 minutes. The supernatant was collected in a new tube and the pellet was discarded. Samples were analyzed by SDS-PAGE (FIG. 8). The yield data is shown in FIG. 7. The two gels show bands at the expected molecular weight of β-casein, even before further purification from endogenous seed proteins. This data demonstrates that proteins like β-casein can be transiently expressed using a variety of plant seeds at detectable levels.

### Example 3: Expressing and Characterizing Kappa-Casein in Plant Seeds

In this example, κ-casein was transiently expressed using pinto bean seeds. A protein expression vector containing a κ-casein transgene was generated based on the native Bos taurus sequence (Uniprot P02668), which was optimized for expression in plants based on the codon usage database (kazusa.jp). A version of the CaMV35S promoter with duplicated enhanced region was selected for this gene construct, the KDEL signal peptide (SEQ ID NO: 19) was added to target the expression towards the secretory pathway. A 10× histidine tag (SEQ ID NO: 20) was added to the N-terminal to facilitate protein purification, and the nopaline synthase (NOS) terminator was added to the construct. The expression cassette was cloned between the sites EcoRI and BamHI of the pCAMBIA2300 vector.

Seeds: Phaseolus vulgaris cv. Pinto Saltillo seeds were obtained at the local food market. The full, intact and healthy seeds were weighed

Bacterium: A. tumefaciens strain GV3101 was transformed with the plasmid bcas-pCAMBIA2300 using heat shock. Once transformed, the culture was incubated for 48 h at 25-30° C. A single colony of A. tumefaciens strain GV3101 containing the plasmid as described above was inoculated in 2-8 mL of LB liquid medium (25-100 μg/mL kanamycin) and was incubated for 48 hours at 25-30° C. at 200 rpm. This pre-inoculum was then transferred to 300-500 mL of fresh LB medium supplemented with kanamycin (25-100 μg/ml). The culture was grown overnight at 25-30° C. at 200 rpm. The cells were harvested by centrifugation at 2000˜3000 rpm for 40-50 minutes at room temperature.

Co-culture: The collected cells were resuspended with infiltration medium (9 mM MES, 10 mM MgCl2, 100 μM acetosyringone) to obtain an optical density (OD) of approximately 0.5-1.5. Subsequently, 100-300 g of seeds were soaked in two volumes (200-600 mL) of the Agrobacterium culture resuspended in MES. Seeds were co-cultured with Agrobacterium for 18-24 h at 25-30° C. in dark conditions. After co-culturing, the seeds were rinsed and incubated during 24 hours with 1-3 volumes of fresh Agrobacterium culture resuspended in MES. In total, three consecutive infections were carried out. After the infections, the seeds were stored at −20° C. to 0° C.

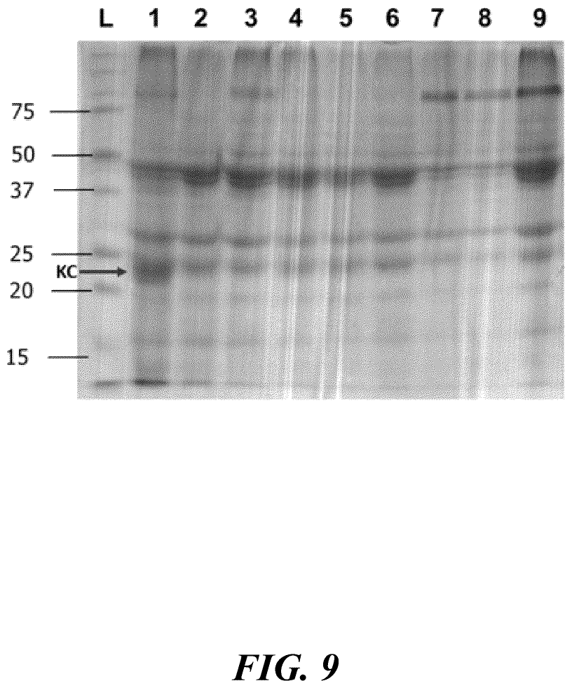

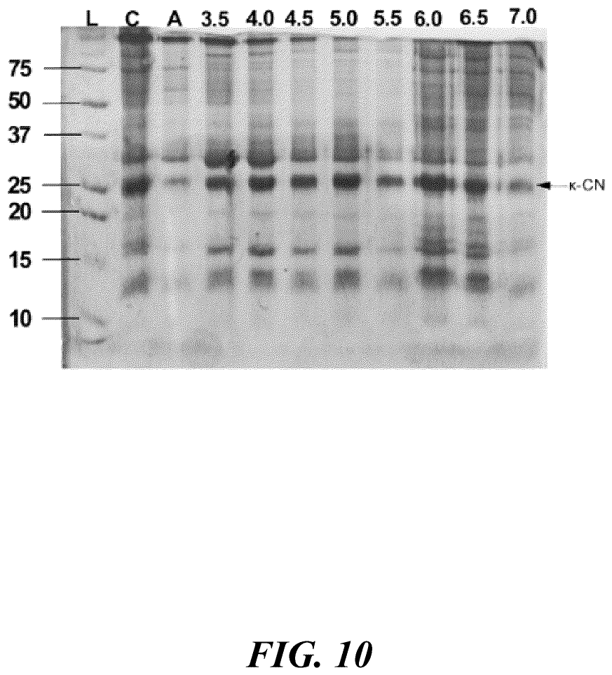

Two different extraction conditions were tested, using either alkaline conditions alone or alkaline conditions followed by acidification. The extraction using alkaline conditions followed the same procedure described in Example 2. An SDS-PAGE analysis of the supernatant was performed and is shown in FIG. 9. The alkaline conditions followed by acidification used the same protocol initially but involved adding 1.4M phosphoric acid to the supernatant to until the pH was equivalent to the isoelectric point of the protein, which was 4.6 in this case. An SDS-PAGE analysis of the acidified supernatant was performed and is shown in FIG. 10. As shown in both FIGS. 9 and 10, bands with the expected molecular weight of κ-casein were observed.

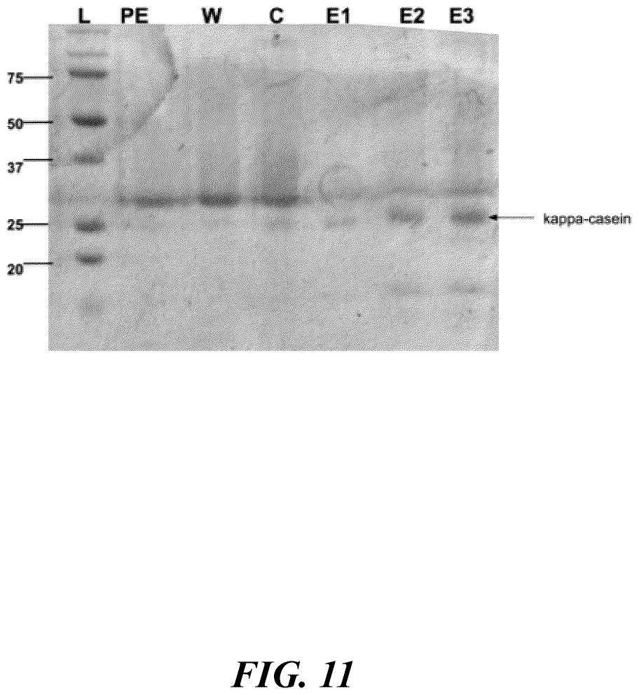

A further His-Tag purification was performed using IMAC and a HisTrap FF (Cytiva) column. The column was equilibrated with 5 volumes Tris-HCl 0.1 M pH 6.8. Then, the sample was loaded into the column. The column was washed with 8-12 volumes of Tris-HCl 0.1 M pH 6.5-7.0 and eluted with a gradient of imidazole between 0 and 500 mM. Fractions containing 0 mM, 50 mM, 100 mM and 500 mM imidazole were collected. Each of the fractions was analyzed using SDS-PAGE and shown in FIG. 11. A band with the expected molecular weight of κ-casein is shown in lanes E1-E3, where E1=50 mM imidazole elution, E2=100 mM imidazole elution and E3=500 mM imidazole elution, demonstrating that κ-casein was produced and isolated using the methods described herein.

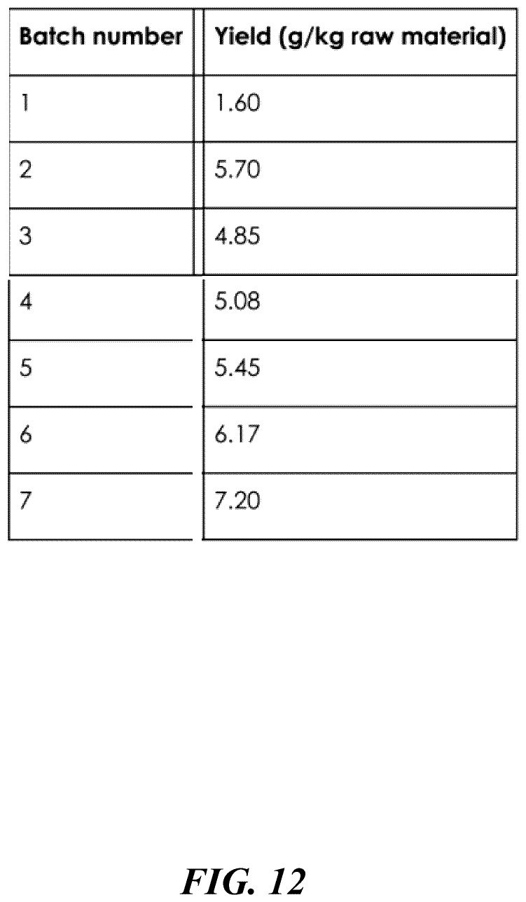

To quantify the expressed protein, image analysis was performed on the gel shown in FIG. 9. Image analysis was performed using ImageJ v1.53s. The original image was converted to an 8 bit-grayscale image with 300 dpi resolution. For the determination of the amount of protein in each band, a comparison of the integrals of color density of each band with a standard BSA curve was carried out. The total volumes for each step and the moisture content before infection (12%) and after the infection (69.7%) were considered for the yield calculations shown in FIG. 12.

Mass spectrometry analysis was performed to further verify that the produced protein was κ-casein. The band corresponding to the eluted material using 500 mM imidazole shown in FIG. 11 was excised from the gel, transferred to a plastic vial and stored at 4° C. In-gel alkylation and reduction was performed by washing the sample with 100-150 μl HPLC grade water for five minutes. The sample was centrifuged, and the liquid discarded. About 3-4× the volume of the gel sample of acetonitrile was added to the tube and incubated for 10-15 minutes. Again, the sample was spun down the liquid was removed and discarded. Gel pieces were incubated in a solution containing 5-15 mM DTT and 30-70 mM ammonium bicarbonate for 20-40 minutes at 50-60° C. Then, gel particles were spun down and the liquid was discarded. Then, 40-70 mM iodoacetamide in 40-60 mM ammonium bicarbonate was added to the gel pieces and the reaction was incubated for 15-25 minutes at room temperature in the dark. Next, iodoacetamide solution was discarded and the gel particles were washed with 150-200 μL of 40-60 mM ammonium bicarbonate for 10-20 minutes. Gel particles were spun down and the liquid was discarded. Gel pieces were shrunk with acetonitrile.

In-gel digestion was performed by rehydrating the gel particles with digestion buffer (50 mM ammonium bicarbonate in HPLC grade water) containing 10-15 ng/uL trypsin at 4° C. (on ice or in a cold room). The mixture was incubated for 30-45 min at 4° C. After 15-20 min, more digestion buffer containing 10-15 ng/uL trypsin to cover the gel particles if they have absorbed the aliquot. The remaining supernatant was discarded. This mixture was incubated at 30-40° C. for 6-12 hours. After incubation, supernatant was recovered in a fresh Eppendorf tube, and 50 μL 2% Acetonitrile 0.1% TFA was added, and the mixture was incubated for 10-20 minutes with agitation. This process was repeated two more times. Next, the supernatants containing the digested peptides were desalted using Thermo Aspire desalting tips.

Resulting peptides were resuspended in 2% acetonitrile 0.1% TFA for LC-MS/MS analysis. Mass spectrometry data was acquired by trapping the peptide sample on a Thermo PepMap trap and separating on an Easy-spray 100 μm×25 cm C18 column using a Dionex Ultimate nUPLC at 200 nL/min. Solvent A=0.1% formic acid and Solvent B=100% acetonitrile with 0.1% formic acid. Gradient conditions were 2% B to 50% B over 60 min, followed by 50% to 99% B in six min held for three min, then 99% B to 2% B in two min for a total run time of 90 min using a Thermo Scientific Fusion Lumos mass spectrometer running in data independent acquisition (DIA) mode.

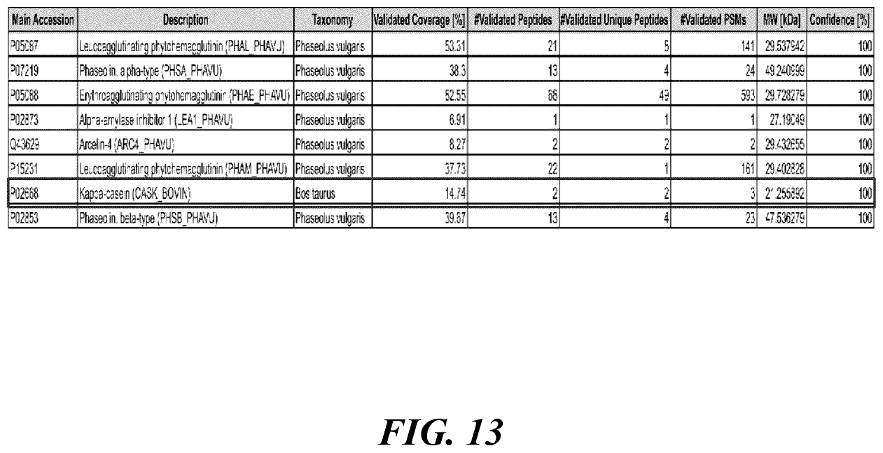

Identification and analysis of the seed product proteins was performed using the PeptideShaker suite v2.2.9. Results of the analysis were filtered, considering positive to the proteins with at least two validated peptide hits and an FDR<1.0. The mass spectrometry results are shown in FIG. 13. As shown, two validated unique peptides associated with κ-casein were identified with 100 confidence. Together, this data, along with the SDS-gel imagery and quantification, demonstrate that κ-casein can be produced in plant seeds using transient expression.

### Example 3: Demonstrating Emulsifying Properties and Making Vegan Cheese Using Kappa-Casein Produced in Plant Seeds

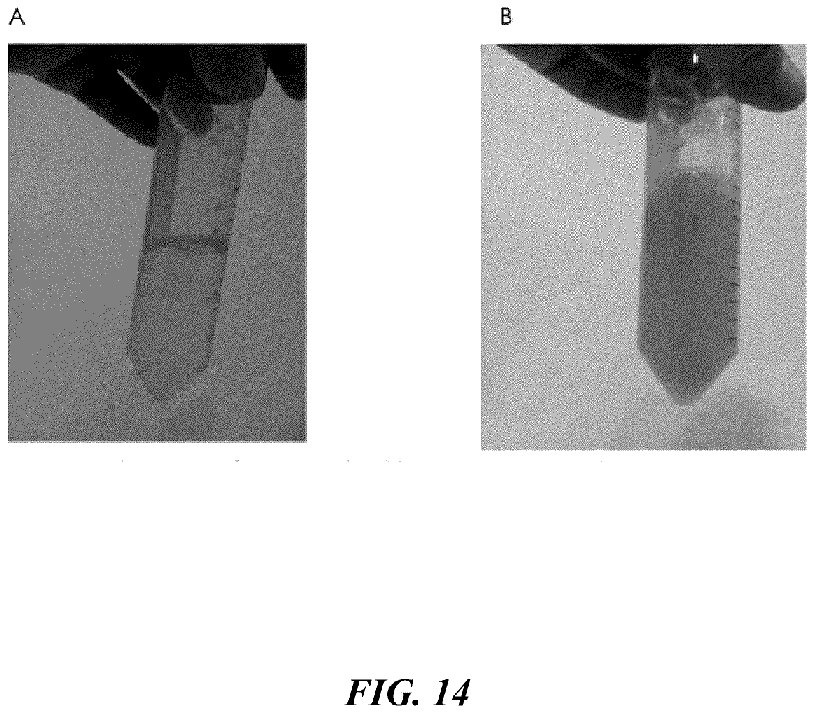

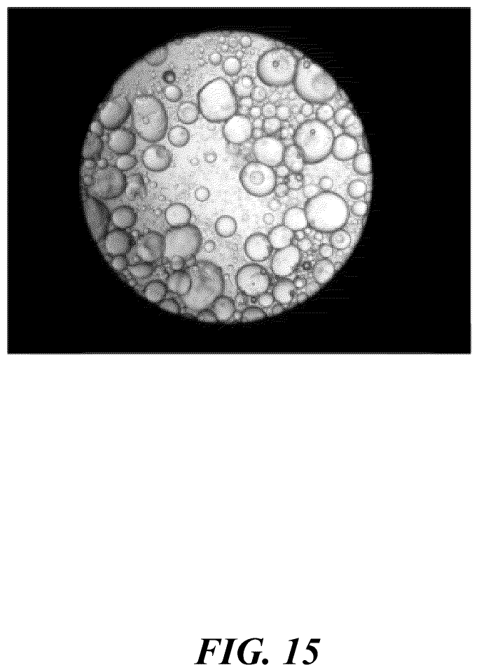

The κ-casein produced according to Example 3 was functionally characterized for emulsification properties. A sample of the isolated protein in dry powder format (1 g) was mixed in a 50-150 ml volume of a 1:1 water-oil mixture. A control sample was also generated without the κ-casein added. Pictures of the two samples are shown in FIG. 14. The control sample pictured in the image on the left clearly shows separated water and oil layers, whereas the sample with the κ-casein added is opaque and well mixed. This demonstrates that the produced κ-casein using the methods described herein can serve as an emulsifying agent, similar to the functional properties of natural κ-casein. To further verify that the mixture depicted on the right in FIG. 14 contains micelles, a sample of the solution was placed on a microscope slide and imaged using a 40× objective lens. The image in FIG. 15 shows the micelles indicative of a water and oil mixture with an emulsifying agent.

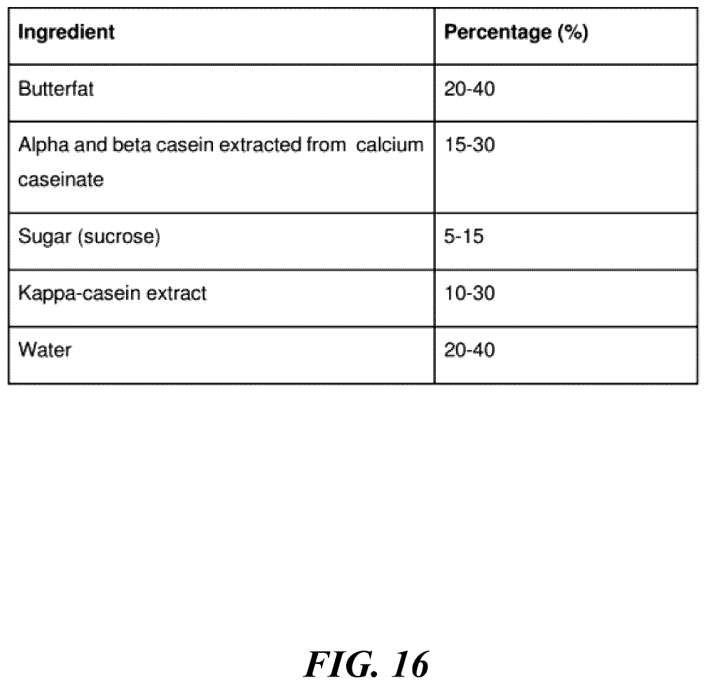

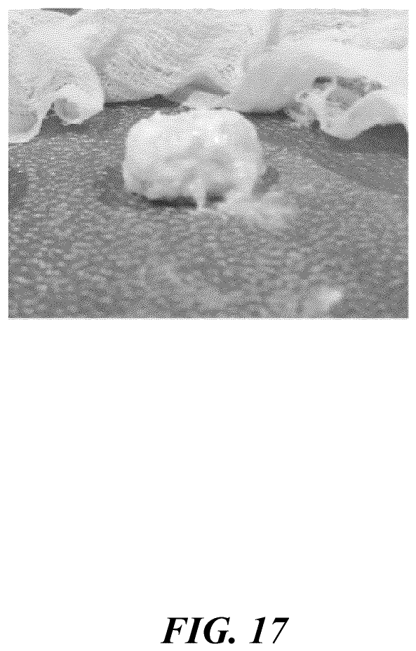

Casein, including κ-casein, is often used for its coagulation properties in cheese formulations. To test whether the produced κ-casein made according to the methods described herein has those characteristics, a vegan cheese formulation was prepared according to the recipe listed in FIG. 16. The cheese was produced by dissolving the butterfat with the seed composition comprising kappa-casein and the water. The mixture was heated for 20-30 seconds until the butterfat was completely dissolved. Then the alpha and beta caseins and the sugar were added. The mixture was homogenized by vortex agitation thoroughly for 20 seconds. The emulsion was adjusted to a pH of 6.8 using phosphoric acid (1.4 M), then acidified again with citric acid to a final pH of 5.2. Vegetable rennet was added and then incubated at 37° C. for 1 hour. The mixture was vortexed again and then heated to 60° C. After reaching the temperature, the structure (cheese) obtained was placed in ice for 1 minute. Then it was drained with cheesecloth overnight at room temperature. A negative control without the seed product comprising kappa-casein was made under the same conditions. An image of the produced coagulated cheese is shown in FIG. 17. As shown, the cheese product is formed and sticks together. This demonstrates that the κ-casein produced by the methods described herein can provide coagulation properties consistent with naturally occurring κ-casein.

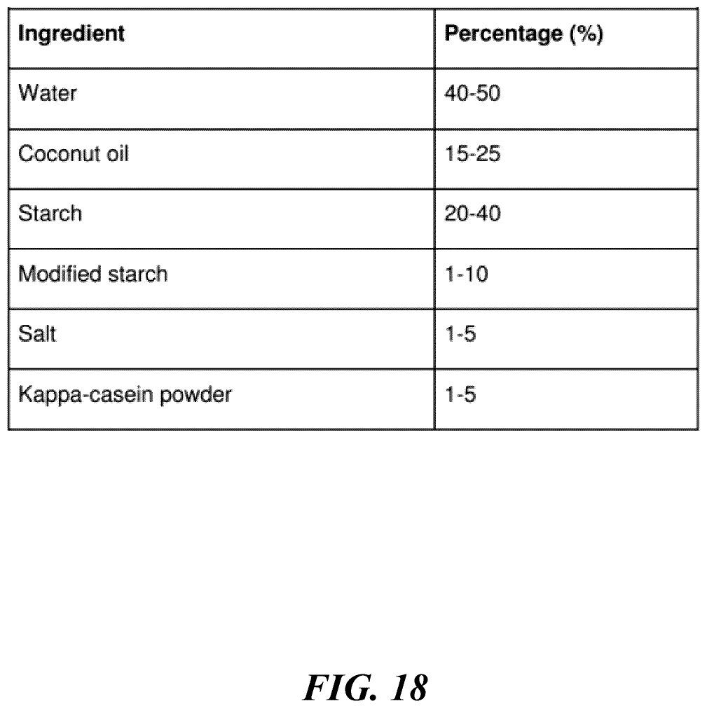

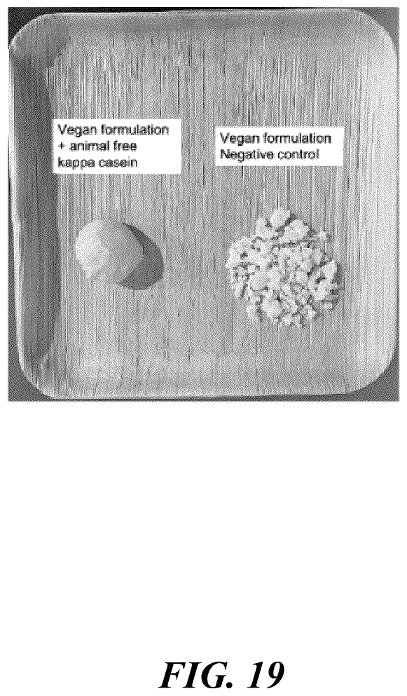

Another key feature of κ-casein is its texturizing properties, including its ability to create a cheese block rather than separate components. Along these lines, another cheese formulation using the κ-casein produced herein was generated according to the recipe in FIG. 18. The κ-casein powder was dissolved with the water and the coconut oil and mixed thoroughly. The remaining ingredients were added. The mixture was heated in a water bath until reaching 70° C. A control sample was produced without the addition of κ-casein powder. The obtained cheese structures were placed on ice for 1 minute, after which it was drained with cheesecloth overnight at room temperature. The resultant product for each condition is shown in FIG. 19. The cheese product produced using the κ-casein addition, resulted in a single structure, whereas the control sample without the κ-casein lacked a cohesive structure and was crumbly in nature. This demonstrates that the κ-casein produced using the methods described herein has the expected texturizing properties of natural κ-casein.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

SEQUENCES

SEQ ID NO: 1: Enhanced promoter region
TGAGACTTTTCAACAAAGGGTAATATCGGGAAACCTCCTCGGATTCCATTGCCCAGC
TATCTGTCACTTCATCAAAAGGACAGTAGAAAAGGAAGGTGGCACCTACAAATGCC
ATCATTGCGATAAAGGAAAGGCTATCGTTCAAGATGCCTCTGCCGACAGTGGTCCCA
AAGATGGACCCCCACCCACGAGGAGCATCGTGGAAAAAGAAGACGTTCCAACCACG
TCTTCAAAGCAAGTGGATTGATGTGATAACATGGTGGAGCACGACACTCTCGTCTAC
TCCAAGAATATCAAAGATACAGTCTCAGAAGACCAAAGGGCTATTGAGACTTTTCA
ACAAAGGGTAATATCGGGAAACCTCCTCGGATTCCATTGCCCAGCTATCTGTCACTT
CATCAAAAGGACAGTAGAAAAGGAAGGTGGCACCTACAAATGCCATCATTGCGATA
AAGGAAAGGCTATCGTTCAAGATGCCTCTGCCGACAGTGGTCCCAAAGATGGACCC
CCACCCACGAGGAGCATCGTGGAAAAAGAAGACGTTCCAACCACGTCTTCAAAGCA
AGTGGATTGATGTGATATCTCCACTGACGTAAGGGATGACGCACAATCCCACTATCC
TTCGCAAGACCTTCCTCTATATAAGGAAGTTCATTTCATTTGGAGAGGACACGCTGA
SEQ ID NO: 2: Signaling peptide
ATGAAATTGTTGATTTTGACTTGCTTGGTTGCTGTTGCTTTGGCT
SEQ ID NO: 3 Signaling peptide
KDELMKLLILTCLVAVALA
SEQ ID NO: 4 Signaling peptide
AAAGATGAATTGATGAAATTCTTCATTTTCACTTGCTTGTTGGCTGTTGCTTTGGC
SEQ ID NO: 5 Signaling peptide
KDELMKFFIFTCLLAVALA
SEQ ID NO: 6 Signaling peptide
AAAGATGAATTGATGAAAGTTTTGATTTTGGCTTGCTTGGTTGCTTTGGCTTTGGCT
SEQ ID NO: 7 Signaling peptide
KDELMKVLILACLVALALA
SEQ ID NO: 8 Signaling peptide
ATGAACTTATGATGAAGTCTTTTTTTCTTGTTGTTACTATTCTTGCTCTTACTCTTCCT
TTTCTTGGTGCT
SEQ ID NO: 9 Signaling peptide
MMKSFFLVVTILALTLPFLGA
SEQ ID NO: 10 Transgene: Alpha-S2 casein
TAAAAATACTATGGAACATGTTTCTTCTTCTGAAGAATCTATTATTTCTCAGGAAACT
TATAAACAGGAAAAAAATATGGCTATTAATCCTTCTAAAGAAAATTTGTGCTCTACT
TTCTGCAAAGAAGTTGTTAGGAATGCTAATGAAGAAGAATATTCTATTGGATCTTCT
TCTGAAGAATCTGCTGAAGTTGCTACTGAAGAAGTTAAAATTACTGTTGATGATAAA
CATTATCAGAAAGCTTTGAATGAAATTAATCAGTTCTATCAGAAATTCCCTCAGTAT
TTGCAGTATTTGTATCAGGGACCTATTGTTTTGAATCCTTGGGATCAGGTTAAAAGG
AATGCTGTTCCTATTACTCCTACTTTGAATAGGGAACAGTTGTCTACTTCTGAAGAA
AATTCTAAAAAAACTGTTGATATGGAATCTACTGAAGTTTTCACTAAAAAAACTAAA
TTGACTGAAGAAGAAAAAAATAGGTTGAATTTCTTGAAAAAAATTTCTCAGAGGTA
TCAGAAATTCGCTTTGCCTCAGTATTTGAAAACTGTTTATCAGCATCAGAAAGCTAT
GAAACCTTGGATTCAGCCTAAAACTAAAGTTATTCCTTATGTTAGGTATTTG
SEQ ID NO: 11 Transgene: Alpha-S2 casein
KNTMEHVSSSEESIISQETYKQEKNMAINPSKENLCSTFCKEVVRNANEEEYSIGSSSEES
AEVATEEVKITVDDKHYQKALNEINQFYQKFPQYLQYLYQGPIVLNPWDQVKRNAVPIT
PTLNREQLSTSEENSKKTVDMESTEVFTKKTKLTEEEKNRLNFLKKISQRYQKFALPQYL
KTVYQHQKAMKPWIQPKTKVIPYVRYL
SEQ ID NO: 12 Transgene: Beta casein
AGGGAATTGGAAGAATTGAATGTTCCTGGAGAAATTGTTGAATCTTTGTCTTCTTCT
GAAGAATCTATTACTAGGATTAATAAAAAAATTGAAAAATTCCAGTCTGAAGAACA
GCAGCAGACTGAAGATGAATTGCAGGATAAAATTCATCCTTTCGCTCAGACTCAGTC
TTTGGTTTATCCTTTCCCTGGACCTATTCCTAATTCTTTGCCTCAGAATATTCCTCCTT
TGACTCAGACTCCTGTTGTTGTTCCTCCTTTCTTGCAGCCTGAAGTTATGGGAGTTTC
TAAAGTTAAAGAAGCTATGGCTCCTAAACATAAAGAAATGCCTTTCCCTAAATATCC
TGTTGAACCTTTCACTGAATCTCAGTCTTTGACTTTGACTGATGTTGAAAATTTGCAT
TTGCCTTTGCCTTTGTTGCAGTCTTGGATGCATCAGCCTCATCAGCCTTTGCCTCCTA
CTGTTATGTTCCCTCCTCAGTCTGTTTTGTCTTTGTCTCAGTCTAAAGTTTTGCCTGTT
CCTCAGAAAGCTGTTCCTTATCCTCAGAGGGATATGCCTATTCAGGCTTTCTTGTTGT
ATCAGGAACCTGTTTTGGGACCTGTTAGGGGACCTTTCCCTATTATTGTT
SEQ ID NO: 13 Transgene: Beta casein
RELEELNVPGEIVESLSSSEESITRINKKIEKFQSEEQQQTEDELQDKIHPFAQTQSLVYPFP
GPIPNSLPQNIPPLTQTPVVVPPFLQPEVMGVSKVKEAMAPKHKEMPFPKYPVEPFTESQ
SLTLTDVENLHLPLPLLQSWMHQPHQPLPPTVMFPPQSVLSLSQSKVLPVPQKAVPYPQ
RDMPIQAFLLYQEPVLGPVRGPFPIIV
SEQ ID NO: 14 Transgene: Kappa casein
CAAGAACAAAATCAAGAACAACCTATTAGATGTGAAAAGGATGAAAGATTTTTTTC
TGATAAGATTGCTAAGTATATTCCTATTCAATATGTTCTTTCTAGATATCCTTCTTAT
GGTCTTAATTATTATCAACAAAAGCCTGTTGCTCTTATTAATAATCAATTTCTTCCTT
ATCCTTATTATGCTAAGCCTGCTGCTGTTAGATCTCCTGCTCAAATTCTTCAATGGCA
AGTTCTTTCTAATACTGTTCCTGCTAAGTCTTGTCAAGCTCAACCTACTACTATGGCT
AGACATCCTCATCCTCATCTTTCTTTTATGGCTATTCCTCCTAAGAAGAATCAAGATA
AGACTGAAATTCCTACTATTAATACTATTGCTTCTGGTGAACCTACTTCTACTCCTAC
TACTGAAGCTGTTGAATCTACTGTTGCTACTCTTGAAGATTCTCCTGAAGTTATTGAA
TCTCCTCCTGAAATTAATACTGTTCAAGTTACTTCTACTGCTGTT
SEQ ID NO: 15 Transgene: Kappa casein
QEQNQEQPIRCEKDERFFSDKIAKYIPIQYVLSRYPSYGLNYYQQKPVALINNQFLPYPY
YAKPAAVRSPAQILQWQVLSNTVPAKSCQAQPTTMARHPHPHLSFMAIPPKKNQDKTEI
PTINTIASGEPTSTPTTEAVESTVATLEDSPEVIESPPEINTVQVTSTAV
SEQ ID NO: 16 Vector sequence
CATGCCAACCACAGGGTTCCCCTCGGGATCAAAGTACTTTGATCCAACCCCTCCGCT
GCTATAGTGCAGTCGGCTTCTGACGTTCAGTGCAGCCGTCTTCTGAAAACGACATGT
CGCACAAGTCCTAAGTTACGCGACAGGCTGCCGCCCTGCCCTTTTCCTGGCGTTTTCT
TGTCGCGTGTTTTAGTCGCATAAAGTAGAATACTTGCGACTAGAACCGGAGACATTA
CGCCATGAACAAGAGCGCCGCCGCTGGCCTGCTGGGCTATGCCCGCGTCAGCACCG
ACGACCAGGACTTGACCAACCAACGGGCCGAACTGCACGCGGCCGGCTGCACCAAG
CTGTTTTCCGAGAAGATCACCGGCACCAGGCGCGACCGCCCGGAGCTGGCCAGGAT
GCTTGACCACCTACGCCCTGGCGACGTTGTGACAGTGACCAGGCTAGACCGCCTGGC
CCGCAGCACCCGCGACCTACTGGACATTGCCGAGCGCATCCAGGAGGCCGGCGCGG
GCCTGCGTAGCCTGGCAGAGCCGTGGGCCGACACCACCACGCCGGCCGGCCGCATG
GTGTTGACCGTGTTCGCCGGCATTGCCGAGTTCGAGCGTTCCCTAATCATCGACCGC
ACCCGGAGCGGGCGCGAGGCCGCCAAGGCCCGAGGCGTGAAGTTTGGCCCCCGCCC
TACCCTCACCCCGGCACAGATCGCGCACGCCCGCGAGCTGATCGACCAGGAAGGCC
GCACCGTGAAAGAGGCGGCTGCACTGCTTGGCGTGCATCGCTCGACCCTGTACCGC
GCACTTGAGCGCAGCGAGGAAGTGACGCCCACCGAGGCCAGGCGGCGCGGTGCCTT
CCGTGAGGACGCATTGACCGAGGCCGACGCCCTGGCGGCCGCCGAGAATGAACGCC
AAGAGGAACAAGCATGAAACCGCACCAGGACGGCCAGGACGAACCGTTTTTCATTA
CCGAAGAGATCGAGGCGGAGATGATCGCGGCCGGGTACGTGTTCGAGCCGCCCGCG
CACGTCTCAACCGTGCGGCTGCATGAAATCCTGGCCGGTTTGTCTGATGCCAAGCTG
GCGGCCTGGCCGGCCAGCTTGGCCGCTGAAGAAACCGAGCGCCGCCGTCTAAAAAG
GTGATGTGTATTTGAGTAAAACAGCTTGCGTCATGCGGTCGCTGCGTATATGATGCG
ATGAGTAAATAAACAAATACGCAAGGGGAACGCATGAAGGTTATCGCTGTACTTAA
CCAGAAAGGCGGGTCAGGCAAGACGACCATCGCAACCCATCTAGCCCGCGCCCTGC
AACTCGCCGGGGCCGATGTTCTGTTAGTCGATTCCGATCCCCAGGGCAGTGCCCGCG
ATTGGGCGGCCGTGCGGGAAGATCAACCGCTAACCGTTGTCGGCATCGACCGCCCG
ACGATTGACCGCGACGTGAAGGCCATCGGCCGGCGCGACTTCGTAGTGATCGACGG
AGCGCCCCAGGCGGCGGACTTGGCTGTGTCCGCGATCAAGGCAGCCGACTTCGTGC
TGATTCCGGTGCAGCCAAGCCCTTACGACATATGGGCCACCGCCGACCTGGTGGAG
CTGGTTAAGCAGCGCATTGAGGTCACGGATGGAAGGCTACAAGCGGCCTTTGTCGT
GTCGCGGGCGATCAAAGGCACGCGCATCGGCGGTGAGGTTGCCGAGGCGCTGGCCG
GGTACGAGCTGCCCATTCTTGAGTCCCGTATCACGCAGCGCGTGAGCTACCCAGGCA
CTGCCGCCGCCGGCACAACCGTTCTTGAATCAGAACCCGAGGGCGACGCTGCCCGC
GAGGTCCAGGCGCTGGCCGCTGAAATTAAATCAAAACTCATTTGAGTTAATGAGGT
AAAGAGAAAATGAGCAAAAGCACAAACACGCTAAGTGCCGGCCGTCCGAGCGCAC
GCAGCAGCAAGGCTGCAACGTTGGCCAGCCTGGCAGACACGCCAGCCATGAAGCGG
GTCAACTTTCAGTTGCCGGCGGAGGATCACACCAAGCTGAAGATGTACGCGGTACG
CCAAGGCAAGACCATTACCGAGCTGCTATCTGAATACATCGCGCAGCTACCAGAGT
AAATGAGCAAATGAATAAATGAGTAGATGAATTTTAGCGGCTAAAGGAGGCGGCAT
GGAAAATCAAGAACAACCAGGCACCGACGCCGTGGAATGCCCCATGTGTGGAGGA
ACGGGCGGTTGGCCAGGCGTAAGCGGCTGGGTTGTCTGCCGGCCCTGCAATGGCAC
TGGAACCCCCAAGCCCGAGGAATCGGCGTGAGCGGTCGCAAACCATCCGGCCCGGT
ACAAATCGGCGCGGCGCTGGGTGATGACCTGGTGGAGAAGTTGAAGGCCGCGCAGG
CCGCCCAGCGGCAACGCATCGAGGCAGAAGCACGCCCCGGTGAATCGTGGCAAGCG
GCCGCTGATCGAATCCGCAAAGAATCCCGGCAACCGCCGGCAGCCGGTGCGCCGTC
GATTAGGAAGCCGCCCAAGGGCGACGAGCAACCAGATTTTTTCGTTCCGATGCTCTA
TGACGTGGGCACCCGCGATAGTCGCAGCATCATGGACGTGGCCGTTTTCCGTCTGTC
GAAGCGTGACCGACGAGCTGGCGAGGTGATCCGCTACGAGCTTCCAGACGGGCACG
TAGAGGTTTCCGCAGGGCCGGCCGGCATGGCCAGTGTGTGGGATTACGACCTGGTA
CTGATGGCGGTTTCCCATCTAACCGAATCCATGAACCGATACCGGGAAGGGAAGGG
AGACAAGCCCGGCCGCGTGTTCCGTCCACACGTTGCGGACGTACTCAAGTTCTGCCG
GCGAGCCGATGGCGGAAAGCAGAAAGACGACCTGGTAGAAACCTGCATTCGGTTAA
ACACCACGCACGTTGCCATGCAGCGTACGAAGAAGGCCAAGAACGGCCGCCTGGTG
ACGGTATCCGAGGGTGAAGCCTTGATTAGCCGCTACAAGATCGTAAAGAGCGAAAC
CGGGCGGCCGGAGTACATCGAGATCGAGCTAGCTGATTGGATGTACCGCGAGATCA
CAGAAGGCAAGAACCCGGACGTGCTGACGGTTCACCCCGATTACTTTTTGATCGATC
CCGGCATCGGCCGTTTTCTCTACCGCCTGGCACGCCGCGCCGCAGGCAAGGCAGAA
GCCAGATGGTTGTTCAAGACGATCTACGAACGCAGTGGCAGCGCCGGAGAGTTCAA
GAAGTTCTGTTTCACCGTGCGCAAGCTGATCGGGTCAAATGACCTGCCGGAGTACGA
TTTGAAGGAGGAGGCGGGGCAGGCTGGCCCGATCCTAGTCATGCGCTACCGCAACC
TGATCGAGGGCGAAGCATCCGCCGGTTCCTAATGTACGGAGCAGATGCTAGGGCAA
ATTGCCCTAGCAGGGGAAAAAGGTCGAAAAGGTCTCTTTCCTGTGGATAGCACGTA
CATTGGGAACCCAAAGCCGTACATTGGGAACCGGAACCCGTACATTGGGAACCCAA
AGCCGTACATTGGGAACCGGTCACACATGTAAGTGACTGATATAAAAGAGAAAAAA
GGCGATTTTTCCGCCTAAAACTCTTTAAAACTTATTAAAACTCTTAAAACCCGCCTG
GCCTGTGCATAACTGTCTGGCCAGCGCACAGCCGAAGAGCTGCAAAAAGCGCCTAC
CCTTCGGTCGCTGCGCTCCCTACGCCCCGCCGCTTCGCGTCGGCCTATCGCGGCCGC
TGGCCGCTCAAAAATGGCTGGCCTACGGCCAGGCAATCTACCAGGGCGCGGACAAG
CCGCGCCGTCGCCACTCGACCGCCGGCGCCCACATCAAGGCACCCTGCCTCGCGCGT
TTCGGTGATGACGGTGAAAACCTCTGACACATGCAGCTCCCGGAGACGGTCACAGC
TTGTCTGTAAGCGGATGCCGGGAGCAGACAAGCCCGTCAGGGCGCGTCAGCGGGTG
TTGGCGGGTGTCGGGGCGCAGCCATGACCCAGTCACGTAGCGATAGCGGAGTGTAT
ACTGGCTTAACTATGCGGCATCAGAGCAGATTGTACTGAGAGTGCACCATATGCGGT
GTGAAATACCGCACAGATGCGTAAGGAGAAAATACCGCATCAGGCGCTCTTCCGCT
TCCTCGCTCACTGACTCGCTGCGCTCGGTCGTTCGGCTGCGGCGAGCGGTATCAGCT
CACTCAAAGGCGGTAATACGGTTATCCACAGAATCAGGGGATAACGCAGGAAAGAA
CATGTGAGCAAAAGGCCAGCAAAAGGCCAGGAACCGTAAAAAGGCCGCGTTGCTG
GCGTTTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAG
CTCCCTCGTGCGCTCTCCTGTTCCGACCCTGCCGCTTACCGGATACCTGTCCGCCTTT
CTCCCTTCGGGAAGCGTGGCGCTTTCTCATAGCTCACGCTGTAGGTATCTCAGTTCG
GTGTAGGTCGTTCGCTCCAAGCTGGGCTGTGTGCACGAACCCCCCGTTCAGCCCGAC
CGCTGCGCCTTATCCGGTAACTATCGTCTTGAGTCCAACCCGGTAAGACACGACTTA
TCGCCACTGGCAGCAGCCACTGGTAACAGGATTAGCAGAGCGAGGTATGTAGGCGG
TGCTACAGAGTTCTTGAAGTGGTGGCCTAACTACGGCTACACTAGAAGGACAGTATT
TGGTATCTGCGCTCTGCTGAAGCCAGTTACCTTCGGAAAAAGAGTTGGTAGCTCTTG
ATCCGGCAAACAAACCACCGCTGGTAGCGGTGGTTTTTTTGTTTGCAAGCAGCAGAT
TACGCGCAGAAAAAAAGGATCTCAAGAAGATCCTTTGATCTTTTCTACGGGGTCTGA
CGCTCAGTGGAACGAAAACTCACGTTAAGGGATTTTGGTCATGCATTCTAGGTACTA
AAACAATTCATCCAGTAAAATATAATATTTTATTTTCTCCCAATCAGGCTTGATCCCC
AGTAAGTCAAAAAATAGCTCGACATACTGTTCTTCCCCGATATCCTCCCTGATCGAC
CGGACGCAGAAGGCAATGTCATACCACTTGTCCGCCCTGCCGCTTCTCCCAAGATCA
ATAAAGCCACTTACTTTGCCATCTTTCACAAAGATGTTGCTGTCTCCCAGGTCGCCGT
GGGAAAAGACAAGTTCCTCTTCGGGCTTTTCCGTCTTTAAAAAATCATACAGCTCGC
GCGGATCTTTAAATGGAGTGTCTTCTTCCCAGTTTTCGCAATCCACATCGGCCAGATC
GTTATTCAGTAAGTAATCCAATTCGGCTAAGCGGCTGTCTAAGCTATTCGTATAGGG
ACAATCCGATATGTCGATGGAGTGAAAGAGCCTGATGCACTCCGCATACAGCTCGA
TAATCTTTTCAGGGCTTTGTTCATCTTCATACTCTTCCGAGCAAAGGACGCCATCGGC
CTCACTCATGAGCAGATTGCTCCAGCCATCATGCCGTTCAAAGTGCAGGACCTTTGG
AACAGGCAGCTTTCCTTCCAGCCATAGCATCATGTCCTTTTCCCGTTCCACATCATAG
GTGGTCCCTTTATACCGGCTGTCCGTCATTTTTAAATATAGGTTTTCATTTTCTCCCAC
CAGCTTATATACCTTAGCAGGAGACATTCCTTCCGTATCTTTTACGCAGCGGTATTTT
TCGATCAGTTTTTTCAATTCCGGTGATATTCTCATTTTAGCCATTTATTATTTCCTTCC
TCTTTTCTACAGTATTTAAAGATACCCCAAGAAGCTAATTATAACAAGACGAACTCC
AATTCACTGTTCCTTGCATTCTAAAACCTTAAATACCAGAAAACAGCTTTTTCAAAG
TTGTTTTCAAAGTTGGCGTATAACATAGTATCGACGGAGCCGATTTTGAAACCGCGG
TGATCACAGGCAGCAACGCTCTGTCATCGTTACAATCAACATGCTACCCTCCGCGAG
ATCATCCGTGTTTCAAACCCGGCAGCTTAGTTGCCGTTCTTCCGAATAGCATCGGTA
ACATGAGCAAAGTCTGCCGCCTTACAACGGCTCTCCCGCTGACGCCGTCCCGGACTG
ATGGGCTGCCTGTATCGAGTGGTGATTTTGTGCCGAGCTGCCGGTCGGGGAGCTGTT
GGCTGGCTGGTGGCAGGATATATTGTGGTGTAAACAAATTGACGCTTAGACAACTTA
ATAACACATTGCGGACGTTTTTAATGTACTGAATTAACGCCGAATTAATTCGGGGGA
TCTGGATTTTAGTACTGGATTTTGGTTTTAGGAATTAGAAATTTTATTGATAGAAGTA
TTTTACAAATACAAATACATACTAAGGGTTTCTTATATGCTCAACACATGAGCGAAA
CCCTATAGGAACCCTAATTCCCTTATCTGGGAACTACTCACACATTATTATGGAGAA
ACTCGAGCTTGTCGATCGACTCTAGCTAGAGGATCGATCCGAACCCCAGAGTCCCGC
TCAGAAGAACTCGTCAAGAAGGCGATAGAAGGCGATGCGCTGCGAATCGGGAGCG
GCGATACCGTAAAGCACGAGGAAGCGGTCAGCCCATTCGCCGCCAAGCTCTTCAGC
AATATCACGGGTAGCCAACGCTATGTCCTGATAGCGGTCCGCCACACCCAGCCGGC
CACAGTCGATGAATCCAGAAAAGCGGCCATTTTCCACCATGATATTCGGCAAGCAG
GCATCGCCATGTGTCACGACGAGATCCTCGCCGTCGGGCATGCGCGCCTTGAGCCTG
GCGAACAGTTCGGCTGGCGCGAGCCCCTGATGCTCTTCGTCCAGATCATCCTGATCG
ACAAGACCGGCTTCCATCCGAGTACGTGCTCGCTCGATGCGATGTTTCGCTTGGTGG
TCGAATGGGCAGGTAGCCGGATCAAGCGTATGCAGCCGCCGCATTGCATCAGCCAT
GATGGATACTTTCTCGGCAGGAGCAAGGTGAGATGACAGGAGATCCTGCCCCGGCA
CTTCGCCCAATAGCAGCCAGTCCCTTCCCGCTTCAGTGACAACGTCGAGCACAGCTG
CGCAAGGAACGCCCGTCGTGGCCAGCCACGATAGCCGCGCTGCCTCGTCCTGGAGT
TCATTCAGGGCACCGGACAGGTCGGTCTTGACAAAAAGAACCGGGCGCCCCTGCGC
TGACAGCCGGAACACGGCGGCATCAGAGCAGCCGATTGTCTGTTGTGCCCAGTCAT
AGCCGAATAGCCTCTCCACCCAAGCGGCCGGAGAACCTGCGTGCAATCCATCTTGTT
CAATCCCCATGGTCGATCGACAGATCTGCGAAAGCTCGAGAGAGATAGATTTGTAG
AGAGAGACTGGTGATTTCAGCGTGTCCTCTCCAAATGAAATGAACTTCCTTATATAG
AGGAAGGTCTTGCGAAGGATAGTGGGATTGTGCGTCATCCCTTACGTCAGTGGAGAT
ATCACATCAATCCACTTGCTTTGAAGACGTGGTTGGAACGTCTTCTTTTTCCACGATG
CTCCTCGTGGGTGGGGGTCCATCTTTGGGACCACTGTCGGCAGAGGCATCTTGAACG
ATAGCCTTTCCTTTATCGCAATGATGGCATTTGTAGGTGCCACCTTCCTTTTCTACTG
TCCTTTTGATGAAGTGACAGATAGCTGGGCAATGGAATCCGAGGAGGTTTCCCGATA
TTACCCTTTGTTGAAAAGTCTCAATAGCCCTTTGGTCTTCTGAGACTGTATCTTTGAT
ATTCTTGGAGTAGACGAGAGTGTCGTGCTCCACCATGTTATCACATCAATCCACTTG
CTTTGAAGACGTGGTTGGAACGTCTTCTTTTTCCACGATGCTCCTCGTGGGTGGGGGT
CCATCTTTGGGACCACTGTCGGCAGAGGCATCTTGAACGATAGCCTTTCCTTTATCG
CAATGATGGCATTTGTAGGTGCCACCTTCCTTTTCTACTGTCCTTTTGATGAAGTGAC
AGATAGCTGGGCAATGGAATCCGAGGAGGTTTCCCGATATTACCCTTTGTTGAAAAG
TCTCAATAGCCCTTTGGTCTTCTGAGACTGTATCTTTGATATTCTTGGAGTAGACGAG
AGTGTCGTGCTCCACCATGTTGGCAAGCTGCTCTAGCCAATACGCAAACCGCCTCTC
CCCGCGCGTTGGCCGATTCATTAATGCAGCTGGCACGACAGGTTTCCCGACTGGAAA
GCGGGCAGTGAGCGCAACGCAATTAATGTGAGTTAGCTCACTCATTAGGCACCCCA
GGCTTTACACTTTATGCTTCCGGCTCGTATGTTGTGTGGAATTGTGAGCGGATAACA
ATTTCACACAGGAAACAGCTATGACATGATTACGAATTCGAGCTCGGTACCCGGGG
ATCCTCTAGAGTCGACCTGCAGGCATGCAAGCTTGGCACTGGCCGTCGTTTTACAAC
GTCGTGACTGGGAAAACCCTGGCGTTACCCAACTTAATCGCCTTGCAGCACATCCCC
CTTTCGCCAGCTGGCGTAATAGCGAAGAGGCCCGCACCGATCGCCCTTCCCAACAGT
TGCGCAGCCTGAATGGCGAATGCTAGAGCAGCTTGAGCTTGGATCAGATTGTCGTTT
CCCGCCTTCAGTTTAAACTATCAGTGTTTGACAGGATATATTGGCGGGTAAACCTAA
GAGAAAAGAGCGTTTATTAGAATAATCGGATATTTAAAAGGGCGTGAAAAGGTTTA
TCCGTTCGTCCATTTGTATGTG
SEQ ID NO: 17 Terminator
CACCATCACCATCACCATCACCATCACCAT
SEQ ID NO: 18 Tag
GATCGTTCAAACATTTGGCAATAAAGTTTCTTAAGATTGAATCCTGTTGCCGGTCTTG
CGATGATTATCATATAATTTCTGTTGAATTACGTTAAGCATGTAATAATTAACATGTA
ATGCATGACGTTATTTATGAGATGGGTTTTTATGATTAGAGTCCCGCAATTATACATT
TAATACGCGATAGAAAACAAAATATAGCGCGCAAACTAGGATAAATTATCGCGCGC
GGTGTCATCTATGTTACTAGATC

What is claimed is:

1. A method for generating a protein product, the method comprising
(a) providing a plant seed;
(b) contacting said plant seed with a protein expression vector that encodes for a protein;
(c) transiently expressing said protein in said plant seed using said protein expression vector; and
(d) isolating said protein from said plant seed prior to a post-germination phase to obtain a protein product, wherein a yield of said protein product is at least about 1 g protein/kg seed.

2. The method of claim 1, wherein the yield of said protein product is at least about 3 g protein/kg seed.

3. The method of claim 1, wherein said plant seed belongs to a Fabaceae family.

4. The method of claim 4, wherein said plant seed that belongs to said Fabaceae family is a dry bean.

5. The method of claim 1, wherein said plant seed belongs to a Poaceae family.

6. The method of claim 1, wherein said plant seed belongs to said Brassicaceae family.

7. The method of claim 1, wherein, in (a), (b), or (c), said plant seed is germinating.

8. The method of claim 1, wherein said plant seed is non-germinating.

9. The method of claim 1, wherein (b) comprises applying a bacteria to said plant seed comprising said protein expression vector.

10. The method of claim 10, wherein said bacteria comprises agrobacteria.

11. The method of claim 1, wherein (b) comprises microbombardment.

12. The method of claim 1, wherein said protein expression vector comprises an intron.

13. The method of claim 1, wherein said protein comprises an antibody.

14. The method of claim 1, wherein said protein comprises an enzyme.

15. The method of claim 1, wherein said protein comprises a dairy protein.

16. The method of claim 15, wherein said dairy protein is a casein protein.

17. The method of claim 16, wherein said casein protein comprises αs1-casein, αs2-casein, β-casein, κ-casein, or a combination thereof.

18. The method of claim 1, wherein said protein comprises an amino acid sequence with at least 80% sequence identity to SEQ ID NO:11.

19. The method of claim 1, wherein said protein comprises an amino acid sequence with at least 80% sequence identity to SEQ ID NO:13.

20. The method of claim 1, wherein said protein comprises an amino acid sequence with at least 80% sequence identity to SEQ ID NO:15.

21. The method of claim 1, wherein said protein comprises insulin.

22. The method of claim 1, wherein said protein comprises a signaling peptide.

23. The method of claim 1, further comprising, in (d), extracting said protein from other proteins in said plant seed.

24. The method of claim 23, wherein extracting said protein comprises his-tag purification.

25. The method of claim 23, wherein extracting said protein comprises ion-exchange chromatography.

26. The method of claim 23, wherein extracting said protein comprises using alkaline conditions.

27. The method of claim 23, wherein extracting said protein comprises using acidic conditions.

28. The method of claim 1, further comprising storing said plant seed at a temperature of between 0° C. and −40° C. after (c) and before (d).


SEQUENCE LISTING (20 sequences)

SEQ ID NO: 1 - DNA, 678 bp, synthetic construct
  source 1..678: mol_type = other DNA
tgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctga

SEQ ID NO: 2 - DNA, 45 bp, synthetic construct
  source 1..45: mol_type = other DNA
atgaaattgttgattttgacttgcttggttgctgttgctttggct

SEQ ID NO: 3 - protein, 19 aa, synthetic construct
KDELMKLLILTCLVAVALA

SEQ ID NO: 4 - DNA, 56 bp, synthetic construct
  source 1..56: mol_type = other DNA
aaagatgaattgatgaaattcttcattttcacttgcttgttggctgttgctttggc

SEQ ID NO: 5 - protein, 19 aa, synthetic construct
KDELMKFFIFTCLLAVALA

SEQ ID NO: 6 - DNA, 57 bp, synthetic construct
  source 1..57: mol_type = other DNA
aaagatgaattgatgaaagttttgattttggcttgcttggttgctttggctttggct

SEQ ID NO: 7 - protein, 19 aa, synthetic construct
KDELMKVLILACLVALALA

SEQ ID NO: 8 - DNA, 71 bp, synthetic construct
  source 1..71: mol_type = other DNA
atgaacttatgatgaagtctttttttcttgttgttactattcttgctcttactcttccttttcttggtgct

SEQ ID NO: 9 - protein, 21 aa, synthetic construct
MMKSFFLVVTILALTLPFLGA

SEQ ID NO: 10 - DNA, 622 bp, synthetic construct
  source 1..622: mol_type = other DNA
taaaaatactatggaacatgtttcttcttctgaagaatctattatttctcaggaaacttataaacaggaaaaaaatatggctattaatccttctaaagaaaatttgtgctctactttctgcaaagaagttgttaggaatgctaatgaagaagaatattctattggatcttcttctgaagaatctgctgaagttgctactgaagaagttaaaattactgttgatgataaacattatcagaaagctttgaatgaaattaatcagttctatcagaaattccctcagtatttgcagtatttgtatcagggacctattgttttgaatccttgggatcaggttaaaaggaatgctgttcctattactcctactttgaatagggaacagttgtctacttctgaagaaaattctaaaaaaactgttgatatggaatctactgaagttttcactaaaaaaactaaattgactgaagaagaaaaaaataggttgaatttcttgaaaaaaatttctcagaggtatcagaaattcgctttgcctcagtatttgaaaactgtttatcagcatcagaaagctatgaaaccttggattcagcctaaaactaaagttattccttatgttaggtatttg

SEQ ID NO: 11 - protein, 207 aa, synthetic construct
KNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 12 - DNA, 627 bp, synthetic construct
  source 1..627: mol_type = other DNA
agggaattggaagaattgaatgttcctggagaaattgttgaatctttgtcttcttctgaagaatctattactaggattaataaaaaaattgaaaaattccagtctgaagaacagcagcagactgaagatgaattgcaggataaaattcatcctttcgctcagactcagtctttggtttatcctttccctggacctattcctaattctttgcctcagaatattcctcctttgactcagactcctgttgttgttcctcctttcttgcagcctgaagttatgggagtttctaaagttaaagaagctatggctcctaaacataaagaaatgcctttccctaaatatcctgttgaacctttcactgaatctcagtctttgactttgactgatgttgaaaatttgcatttgcctttgcctttgttgcagtcttggatgcatcagcctcatcagcctttgcctcctactgttatgttccctcctcagtctgttttgtctttgtctcagtctaaagttttgcctgttcctcagaaagctgttccttatcctcagagggatatgcctattcaggctttcttgttgtatcaggaacctgttttgggacctgttaggggacctttccctattattgtt

SEQ ID NO: 13 - protein, 209 aa, synthetic construct
RELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 14 - DNA, 507 bp, synthetic construct
  source 1..507: mol_type = other DNA
caagaacaaaatcaagaacaacctattagatgtgaaaaggatgaaagatttttttctgataagattgctaagtatattcctattcaatatgttctttctagatatccttcttatggtcttaattattatcaacaaaagcctgttgctcttattaataatcaatttcttccttatccttattatgctaagcctgctgctgttagatctcctgctcaaattcttcaatggcaagttctttctaatactgttcctgctaagtcttgtcaagctcaacctactactatggctagacatcctcatcctcatctttcttttatggctattcctcctaagaagaatcaagataagactgaaattcctactattaatactattgcttctggtgaacctacttctactcctactactgaagctgttgaatctactgttgctactcttgaagattctcctgaagttattgaatctcctcctgaaattaatactgttcaagttacttctactgctgtt

SEQ ID NO: 15 - protein, 169 aa, synthetic construct
QEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 16 - DNA, 8743 bp, synthetic construct
  source 1..8743: mol_type = other DNA
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagaggatcgatccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgtgtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccccatggtcgatcgacagatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtg

SEQ ID NO: 17 - DNA, 30 bp, synthetic construct
  source 1..30: mol_type = other DNA
caccatcaccatcaccatcaccatcaccat

SEQ ID NO: 18 - DNA, 253 bp, synthetic construct
  source 1..253: mol_type = other DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 19 - protein, 4 aa, synthetic construct
KDEL

SEQ ID NO: 20 - protein, 10 aa, synthetic construct
HHHHHHHHHH